SEQUENCE LISTING (23 sequences)

SEQ ID NO: 1 - protein, 90 aa, Bacteroides sp.
MNKADLISAVAAEAGLSKVDAKKAVEAFVSTVTKALQEGDKVSLIGFGTFSVAERSARTGINPSTKATITIPAKKVTKFKPGAELADAIK

SEQ ID NO: 2 - DNA, 13 bp, Bacteroides sp.
cctgtctctccgc

SEQ ID NO: 3 - DNA, 11383 bp, Artificial Sequence
  Description of Artificial Sequence Synthetic polynucleotide
ggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcccttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatattcgtttaatatcataaataatttattttattttaaaatgcgcgggtgcaaaggtaagaggttttattttaactaccaaatgttttcggaagttttttcgcttttctttttctatcgtttctcagactctcttagcgaaagggaaagaaggtaaagaagaaaaacaaaacgccttttcttttttgcacccgctttccaagagaagaaagccttgttaaattgacttagtgtaaaagcgcagtactgcttgaccataagaacaaaaaaatctctatcactgatagggataaagtttggaagataaagctaaaagttcttatctttgcagtctccctatcagtgatagagacgaaataaagacatataaaagaaaagacaccatggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacggtggaggaggttctggaggtggaggttctgctgagtatgtgcgagccctctttgactttaatgggaatgatgaagaggatcttccctttaagaaaggagacatcctgagaatccgggataagcctgaggagcagtggtggaatgcagaggacagcgaaggaaagagggggatgattcctgtcccttacgtggagaagtattccggagactataaggaccacgacggagactacaaggatcatgatattgattacaaagacgatgacgataagtctaggctcgagtccggagactataaggaccacgacggagactacaaggatcatgatattgattacaaagacgatgacgataagtctaggatgaccgacgctgagtacgtgagaatccatgagaagttggacatctacacgtttaagaaacagtttttcaacaacaaaaaatccgtgtcgcatagatgctacgttctctttgaattaaaacgacggggtgaacgtagagcgtgtttttggggctatgctgtgaataaaccacagagcgggacagaacgtggcattcacgccgaaatctttagcattagaaaagtcgaagaatacctgcgcgacaaccccggacaattcacgataaattggtactcatcctggagtccttgtgcagattgcgctgaaaagatcttagaatggtataaccaggagctgcgggggaacggccacactttgaaaatctgggcttgcaaactctattacgagaaaaatgcgaggaatcaaattgggctgtggaatctcagagataacggggttgggttgaatgtaatggtaagtgaacactaccaatgttgcaggaaaatattcatccaatcgtcgcacaatcaattgaatgagaatagatggcttgagaagactttgaagcgagctgaaaaacgacggagcgagttgtccattatgattcaggtaaaaatactccacaccactaagagtcctgctgtttaaattaatgcggctgcaatttttttgggcggggccgcccaaaaaaatcctagcaccctgcagcagtactgcttgaccataagaacaaaaaaacttccgataaagtttggaagataaagctaaaagttcttatctttgcagtatacaagagaccagaagaaggttttagagctagaaatagcaagttaaaataaggctagtccgttatcaacttgaaaaagtggcaccgagtcggtgctttttttgagatctgtcgactctagaggatccccgggtaccgagctcgaattcactggccgtcgttttacaacgtcgtgactgggaaaaccctggcgttacccaacttaatcgtacttgtgcctgttctatttccgaaccgaccgcttgtatgaatccatcaaaattcgttttctctatgttggattccttgttgctcatattgtgatgataatttctacaaatatagtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtccttcttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcgctggtgtctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgctctttacccgctgcttatctgcatcccatttggctacgtcaatccggtagcccgttgtaaactcgatgcgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcggctgtttttgttttatttaagataatacaaccactacataataaaagagtagcgatattaaaagaatccgatgagaaaagactaatatttatctatccattcagtttgatttttcaggactttacatcgtcctgaaagtatttgttggtaccggtaccgaggacgcgtaaacatttacagttgcatgtggcctattgtttttagccgttaaatattttataactattaaatagcgatacaaattgttcgaaactaatattgtttatatcatatattctcgcatgttttaaagctttattaaattgattttttgtaaacagtttttcgtactctttgttaacccatttcattacaaaagtttcatatttttttctctctttaaatgccatttttgctggctttctttttaatacaattaatgtgctatccactttaggttttggatggaaataatacctaggaatttttgctaatatagaaatatctacctctgccattaacagcaatgctagtgatctgtttgtatctaataacattttagcaaaaccatattccactattaaataacttattgtggctgaactttcaaaaacaatttttcgaattatatttgtgcttatgttgtaaggtatgctgccaaatattttatatggattgtggctaggaaatgtaaatttcagtatatcatcatttactatttgatagttaggataatttaagagcttattacgagttacctcacataatttagaatcaatttctatcgccgttacaaaattacatctctttaccaatccagcagtaaaatgacctttccctgcacctatttcaaagatgttatctttttcatctaaacttatgcaattcattattttttctatgtgatattttgaagtaataaaattttgactatcttttatatttactttgttcattataacctctccttaatttattgcatctcttttcgaatatttatgttttttgagaaaagaacgtactcatggttcatcccgatatgcgtatcggtctgtatatcagcaactttctatgtgtttcaactacaatagtcatctattctcatctttctgagtccaccccctgcaaagcccctctttacgacataaaaattcggtcggaaaaggtatgcaaaagatgtttctctctttaagagaaactcttcgggatgcaaaaatatgaaaataactccaattcaccaaattatatagcgacttttttacaaaatgctaaaatttgttgatttccgtcaagcaattgttgagcaaaaatgtcttttacgataaaatgatacctcaatatcaactgtttagcaaaacgatatttctcttaaagagagaaacacctttttgttcaccaatccccgacttttaatcccgcggccatgattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcataacgcgtcaattcgagggggatcaattccgtgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaacggaattgatccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattcccctccaccgcggtgg

SEQ ID NO: 4 - DNA, 20 bp, Artificial Sequence
  Description of Artificial Sequence Synthetic oligonucleotide
tgatggagaggtgcaagtag

SEQ ID NO: 5 - DNA, 20 bp, Bacteroides thetaiotaomicron
atacaagagaccagaagaag

SEQ ID NO: 6 - DNA, 20 bp, Bacteroides thetaiotaomicron
gctcaaatccgtattcgtgg

SEQ ID NO: 7 - DNA, 13307 bp, Artificial Sequence
  Description of Artificial Sequence Synthetic polynucleotide
tcgggacgctcatcaatatccaccctgcctgggataaatcctcgccctgcatttttagaaccacgtttggcatacctgcgaccttgtctgcgaagatatttgtgcagtttgccaccccgccgcttatcctcccaaatccagcgatatatcgtttcgtgagataccatcgcaattccctccaagcggctcctgccgacaatctgctccgggctgaatcctttcttcaacagctttattatccgttttctcattgccggtgtaagcacttccttgcgatgtttttgctgcttgcgcctgtctgcttttcgctgggcaagctccatgctatagctaccacttcgggcgtcgcaattgcgctttatctccctgtaaacagtgcttttatctactccgatagcttccgctattgcttttttgctcatcggtatttgcaacatcatagaaattgcatacctttgttcctcggttatatgtttgctcatctgcaacttttttttctttggacggacaattaaagcaaagatagcaaactttatccattcagagtgagagaaagggggacattgtctctctttcctctctgaaaaataaatgtttttattgcttattatccgcacccaaaaagttgcatttataagttgaactcaagaagtattcacctgtaagaagttactaatgacaaaaaagaaattgcccgttcgttttacgggtcagcactttactattgataaagtgctaataaaagatgcaataagacaagcaaatataagtaatcaggatacggttttagatattggggcaggcaaggggtttcttactgttcatttattaaaaatcgccaacaatgttgttgctattgaaaacgacacagctttggttgaacatttacgaaaattattttctgatgcccgaaatgttcaagttgtcggttgtgattttaggaattttgcagttccgaaatttcctttcaaagtggtgtcaaatattccttatggcattacttccgatattttcaaaatcctgatgtttgagagtcttggaaattttctgggaggttccattgtccttcaattagaacctacacaaaagttattttcgaggaagctttacaatccatataccgttttctatcatactttttttgatttgaaacttgtctatgaggtaggtcctgaaagtttcttgccaccgccaactgtcaaatcagccctgttaaacattaaaagaaaacacttattttttgattttaagtttaaagccaaatacttagcatttatttcctgtctgttagagaaacctgatttatctgtaaaaacagctttaaagtcgattttcaggaaaagtcaggtcaggtcaatttcggaaaaattcggtttaaaccttaatgctcaaattgtttgtttgtctccaagtcaatggttaaactgttttttggaaatgctggaagttgtccctgaaaaatttcatccttcgtagttcaaagtcgggtggttgtcaagatgatttttttggtttggtgtcgtctttttttaagctgccgcataacggctggcaaattggcgatggagccgactttggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatccttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcccttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatattcgtttaatatcataaataatttattttattttaaaatgcgcgggtgcaaaggtaagaggttttattttaactaccaaatgttttcggaagttttttcgcttttctttttctatcgtttctcagactctcttagcgaaagggaaagaaggtaaagaagaaaaacaaaacgccttttcttttttgcacccgctttccaagagaagaaagccttgttaaattgacttagtgtaaaagcgcagtactgcttgaccataagaacaaaaaaatctctatcactgatagggataaagtttggaagataaagctaaaagttcttatctttgcagtctccctatcagtgatagagacgaaataaagacatataaaagaaaagacaccatggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacggtggaggaggttctggaggtggaggttctgctgagtatgtgcgagccctctttgactttaatgggaatgatgaagaggatcttccctttaagaaaggagacatcctgagaatccgggataagcctgaggagcagtggtggaatgcagaggacagcgaaggaaagagggggatgattcctgtcccttacgtggagaagtattccggagactataaggaccacgacggagactacaaggatcatgatattgattacaaagacgatgacgataagtctaggctcgagtccggagactataaggaccacgacggagactacaaggatcatgatattgattacaaagacgatgacgataagtctaggatgaccgacgctgagtacgtgagaatccatgagaagttggacatctacacgtttaagaaacagtttttcaacaacaaaaaatccgtgtcgcatagatgctacgttctctttgaattaaaacgacggggtgaacgtagagcgtgtttttggggctatgctgtgaataaaccacagagcgggacagaacgtggcattcacgccgaaatctttagcattagaaaagtcgaagaatacctgcgcgacaaccccggacaattcacgataaattggtactcatcctggagtccttgtgcagattgcgctgaaaagatcttagaatggtataaccaggagctgcgggggaacggccacactttgaaaatctgggcttgcaaactctattacgagaaaaatgcgaggaatcaaattgggctgtggaatctcagagataacggggttgggttgaatgtaatggtaagtgaacactaccaatgttgcaggaaaatattcatccaatcgtcgcacaatcaattgaatgagaatagatggcttgagaagactttgaagcgagctgaaaaacgacggagcgagttgtccattatgattcaggtaaaaatactccacaccactaagagtcctgctgtttaaattaatgcggctgcaatttttttgggcggggccgcccaaaaaaatcctagcaccctgcagcagtactgcttgaccataagaacaaaaaaacttccgataaagtttggaagataaagctaaaagttcttatctttgcagttgatggagaggtgcaagtaggttttagagctagaaatagcaagttaaaataaggctagtccgttatcaacttgaaaaagtggcaccgagtcggtgctttttttgtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacacaccataaactttttttagaataagcacacaaccgttttccgaaccctgcaaaatgttttctgaatccgaacggtgtaacactccattgagagaggctgccgtttggtcgctccccctttgggggcgggggggggttacatacccatgccgaaacctctgcttctggtgatttgcttgaataggtctttcccctcttccatagcttttgatatgtttgggaaatgatgccttaaagcctccagttgttcggaattgaacaagtctttcatcttaccaagttcttttttcaactccttggtttcggcttttagtttttggttctccgtccttaataggttactggttgtccttgcgttgtccatttgttgtctataatactccttgtcattctcggctttgaatgcctttgtgctgtttcgctctttttcaagtatagcctttcccagtctatcggatagttgttcattttccccctctaaagtctttactttggcttttaaggcatccttttccctatcgttgactgtttttccaatcaagccgtaaaacttctctgaagccttagaaatgagtttttggacgttcttctttgtttcaatggaacgtagttccttctgaagctgaagaagctggttttgtgcgtccttgtatttgtctaatgcactggatatatcgttggatagttcctgaagctgttctttcgcacattcggtcttgtactgcatagccgataagtgtttgcggtcagaagaaacgccacgttccatgcccagtgtttcagatgctatggtttggagttctgccatgtcatcacgcgataaacgcacacttttcccattcggctgcgtccaatcgaaaactacatgggcatgaaggttaggtgtccactgctttgcgttcatgtatccttcgtccttgtgtatatggatttgaaacgcttcgataccgaaacgttctttgcagaccgtggcaaactgctggagttcctgcatagtggtttcttgtttgattactattactccctctcgtatgggtgcggctttagcctgcatcttctgcccaaccgtatcgagatatctttgttttgcactctccagccgatgggaaatgctatctccaacccagctttcattcaaatgactaagttcgggacgaacatagtccaactctttttccctaaagttgtgaatctcgctccccggcttcactgcttgtacatgaatacttgttgctcccataagttaacatttttgtgacaatcgataacagccggtgacagccggctgacagggggttaagggggcttgtccccttacacacgcactctttagggtgctagtgtgctatcaccatactgcataggtgcgaagttagtgaatgttttgtaaatgcacaaataaagggaaaaacatttggatttgcgataataaagtactacctttgttgctgaccaaacggtagctgaccgatacgggagagttaccaaaatacaagccgctggagttaattgacggacatccgacatctccagcggctttatttttgcctatctgcttcgcctaggcacaccagtacctctactaaaaatgtacttcaaagatacttattttctaccgacttgatagtttttaccccatattcttggacatttttcccccatgaggttatctttgtagggtgaaagagaaacccataaacggggatagattgaatgctgggaagcataaacaatcggggtaaggttagcgaaccttgcctttcatcccccattataactttacatagaggaactttatctatccccccccgcccccaaagggggagcgaccaaacggcagcttcactcaatggagtgttactgttcatcaaagccaagtgataattgtcgtttctctgcttcttctttcttttgggcagctaaagtctttttccgaacgtatgttttagcaaatgtcactcggtcaccattgaatactatcagaggattaataaaccaaagattatcggctggtcctcgggctatgatttcagcttttacaagttctgcaagtcctttataaacggctttgtctgttttgtatttggtatattctaggcatttttttctattgaaaatgattaaatcatttttgggtttcatgcaggtcataaagtaaccaaaaacccgaatagctgcttgtgataggtcaaagaatgcagcaaagttagaaagatacaatttagtgaattgttcttcatctacttctatttgacggataaacgaagtcttaaacacttctccagtttcagtgtcggctaaagctactacagctctcttatcgccaccactattactcttatactttttaacaacatgattttcaataccttctatagcttgtttcataaaaggattttcttcgttcttttgaaaatcggttaacttaactgcttttttattttccattttgatatgtttttgggaaatattattctccacaaagtaaactattattttccataaaaacaatattaagggaaatattattttcctatttagtatcatattaggaaatcggtattttctagattggaaaatgagaatttccaatatggaaaatgccctatattgtgtatcaagtacttaacttattctatttcttttattcttaatatacccccaaaacagcacaaaatcagtcacttaaaaatcatcggtcggggaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagctaaatttaaatataaacaa

SEQ ID NO: 8 - DNA, 20 bp, Bacteroides thetaiotaomicron
ggacgaatcgtaaatgcaga

SEQ ID NO: 9 - DNA, 20 bp, Bacteroides thetaiotaomicron
cccattggctgaatgtggcg

SEQ ID NO: 10 - DNA, 51 bp, Bacteroides thetaiotaomicron
  CDS (1)..(51)
atggtattattttcagaagaccacatacaagagaccagaagaagaggtaga

SEQ ID NO: 11 - protein, 17 aa, Bacteroides thetaiotaomicron
MVLFSEDHIQETRRRGR

SEQ ID NO: 12 - DNA, 51 bp, Artificial Sequence
  Description of Artificial Sequence Synthetic oligonucleotide
  CDS (1)..(27)
atggtattattttcagaagaccacatataagagaccagaagaagaggtaga

SEQ ID NO: 13 - protein, 9 aa, Artificial Sequence
  Description of Artificial Sequence Synthetic peptide
MVLFSEDHI

SEQ ID NO: 14 - DNA, 36 bp, Bacteroides thetaiotaomicron
  CDS (1)..(36)
ggtggtggcgctcaaatccgtattcgtggcggttct

SEQ ID NO: 15 - protein, 12 aa, Bacteroides thetaiotaomicron
GGGAQIRIRGGS

SEQ ID NO: 16 - DNA, 36 bp, Artificial Sequence
  Description of Artificial Sequence Synthetic oligonucleotide
  CDS (1)..(12)
ggtggtggcgtttaaatccgtattcgtggcggttct

SEQ ID NO: 17 - protein, 4 aa, Artificial Sequence
  Description of Artificial Sequence Synthetic peptide
GGGV

SEQ ID NO: 18 - DNA, 80 bp, Bacteroides thetaiotaomicron
tgggcgcaagatgctaaagtcctgaaaggacgaatcgtaaatgcagagggtgaacctatcgcaggtgcggtggtcaacgt

SEQ ID NO: 19 - DNA, 80 bp, Artificial Sequence
  Description of Artificial Sequence Synthetic oligonucleotide
tgggcgcaagatgctaaagtcctgaaaggatgaatcgtaaatgcagagggtgaacctatcgcaggtgcggtggtcaacgt

SEQ ID NO: 20 - DNA, 81 bp, Bacteroides thetaiotaomicron
tgggcgcaagatgctaaagtcctgaaaggacgaatcgtaaatgcagagggtgaacctatcgcaggtgcggtggtcaacgta

SEQ ID NO: 21 - DNA, 80 bp, Bacteroides thetaiotaomicron
cggtgatctgcttgttctgcgcatacacccattggctgaatgtggcgaggcagagtatcagtataaaatttttcatgtat

SEQ ID NO: 22 - DNA, 80 bp, Artificial Sequence
  Description of Artificial Sequence Synthetic oligonucleotide
ccgtccggtgatctgcttgttctgcgcatacatttattggctgaatgtggcgaggcagagtatcagtataaaatttttca

SEQ ID NO: 23 - DNA, 81 bp, Bacteroides thetaiotaomicron
ccgtccggtgatctgcttgttctgcgcatacacccattggctgaatgtggcgaggcagagtatcagtataaaatttttcat

(19) United States
(12) Patent Application Publication
(10) Pub. No.: US 2021/0180071 A1
(43) Pub. Date: Jun. 17, 2021

(54) GENOME EDITING IN BACTEROIDES

(71) Applicant: SIGMA-ALDRICH CO. LLC, St. Louis, MO (US)

(72) Inventors: Erik Eastlund, Fenton, MO (US); Zhigang Zhang, Creve Coeur, MO (US); Gregory D. Davis, Webster Groves, MO (US)

(21) Appl. No.: 17/125,456

(22) Filed: Dec. 17, 2020

Related U.S. Application Data
(60) Provisional application No. 62/949,314, filed on Dec. 17, 2019.

(51) Int. Cl. C12N 15/74; C12N 9/22; C12N 15/11

(52) U.S. Cl. CPC C12N 15/74; C12N 9/22; C12N 2310/16; C12N 2310/20; C12N 2800/80; C12N 15/11

(57) ABSTRACT

Compositions and methods for genome editing of Bacteroides species are provided herein. RNA-guided nucleobase modification systems are engineered to target specific loci in chromosomal DNA of a target bacteria cell, wherein the genome of the target bacterial cell can be modified.

### CROSS-REFERENCE TO RELATED APPLICATIONS

The present application claims the benefit of priority of U.S. Provisional Application No. 62/949,314, filed Dec. 17, 2019, the entire contents of which is incorporated herein by reference.

### SEQUENCE LISTING

The instant application contains a Sequence Listing that has been submitted in ASCII format via EFS-Web and is hereby incorporated by reference in its entirety. The ASCII copy, created on Dec. 17, 2020, is named P19-235_US-NP_SL.txt, and is 38,714 bytes in size.

### FIELD

The present disclosure relates to compositions and methods for genome editing in Bacteroides.

### BACKGROUND

Controlling the ability to specifically modify DNA sequences in a microbial genome is a critical aspect of medicine and biotechnology research. Recent advances indicate that RNA-guided systems can be designed to target specific DNA sequences in microbial genomes, however, the unique DNA repair status and molecular epigenetic structure in which various microbial genomes exist creates uncertainty about the effectiveness of particular genome editing technologies. Here we describe compositions and methods which are effective for modifying genomes of Bacteroides species.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The patent or application file contains at least one drawing executed in color. Copies of this patent or patent application publication with color drawing(s) will be provided by the Office upon request and payment of the necessary fee.

FIG. 1 presents a schematic model for CRISPR base editing (dSpCas9-CDA/sgRNA). The dSpCas9-CDA/sgRNA complex binds to the double-stranded DNA to form an R-loop in a sgRNA- and PAM-dependent manner. CDA catalyzes deamination of cytosines located at the bottom (non-complementary) strand within 15-20 bases upstream from the PAM, which results in C-to-T mutagenesis.

FIG. 2 presents a schematic of a CRISPR base editor integration plasmid [pNBU2.CRISPR-CDA] targeting tdk (BT_2275) in Bacteroides thetaiotaomicron.

FIG. 3A shows sequence alignment of the tdk_Bt mutants edited by dSpCas9-CDA. The genomic loci and the site targeted by tdk_Bt sgRNA (N20) are shown with a PAM. The coding sequence of tdk_Bt is shown on the top, beginning at the ATG start codon. Mutated sites found from eight randomly picked colonies from aTc100 agar plates are shown on the bottom. The mutated base (C to T at position −17 from the PAM) resulted in a stop codon at position 28 of the tdk_Bt coding sequence. FIG. 3A discloses SEQ ID NOS 10-13, respectively, in order of appearance.

FIG. 3B presents sequence alignment of the susC_Bt mutants edited by dSpCas9-CDA. The genomic loci and the site targeted by susC_Bt sgRNA (N20) are shown with a PAM. The coding sequence of susC_Bt is shown on the top. Mutated sites found from eight randomly picked colonies from aTc100 agar plates are shown on the bottom. The mutated bases (C to T at positions −17 and −19 from the PAM) generate an amino acid substitution and a stop codon at positions 491 and 493 of the susC_Bt coding sequence. FIG. 3B discloses SEQ ID NOS 14-17, respectively, in order of appearance.

FIG. 4 presents a schematic of a CRISPR base editor stably maintained plasmid (pmobA.repA.CRISPR-CDA.NT) with a non-targeting guide RNA scrambled nucleotide sequence that does not target the Bacteroides thetaiotaomicron VPI-5482 genome.

FIG. 5A shows 25 μg/ml erythromycin (Em) and 200 μg/ml gentamicin (Gm) brain-heart infusion (BHI) blood agar plates that were plated with 100 μl of a 1:10 dilution from reconstituted 1 ml aerobic E. coli/Bacteroides thetaiotaomicron VPI-5482 conjugation slurries. These reconstituted conjugation slurries were from no selection BHI blood agar plates. Plates from left to right show the non-targeting sample, the BT_0362 sample and the BT_0364 sample.

FIG. 5B shows sterile loop growth streaks on 25 μg/ml Em, 200 μg/ml Gm and 100 ng/ml anhydrotetracycline (aTc) selection and induction BHI blood agar plates. Individual colonies from each plate shown in FIG. 5A were grown in 5 ml of selection and induction TYG liquid medium supplemented with 25 μg/ml Em, 200 μg/ml Gm and 100 ng/ml aTc. The sterile loop samples were taken from these selection and induction TYG liquid media cultures. Plates from left to right show the non-targeting sample, the BT_0362 sample and the BT_0364 sample.

FIG. 6A illustrates quantitative mutational analysis using MilliporeSigma internally developed software called “SangerTrace”. This analysis software extracts each base signal peak value, based on Applied Biosystem's, Inc. format (ABI) file, and calculates mutation percentages by comparing “control” and “sample” Sanger sequencing data. The top Sanger trace is the non-targeting sample with the guide RNA sequence underlined. The red arrow shows base −17, relative to the PAM, that is the location of the cytosine deamination, which leads to C-to-T mutagenesis and the introduction of a stop codon truncating the BT_0362 coding sequence. The middle Sanger trace shows the BT_0362 edited sample and the lower graph shows the C-to-T mutation frequency. FIG. 6A discloses SEQ ID NOS 18-20, respectively, in order of appearance.

FIG. 6B illustrates quantitative mutational analysis using MilliporeSigma internally developed software called “SangerTrace”. This analysis software extracts each base signal peak value, based on Applied Biosystem's, Inc. format (ABI) file, and calculates mutation percentages by comparing “control” and “sample” Sanger sequencing data. The top Sanger trace is the non-targeting sample with the guide RNA sequence underlined. The red arrow shows bases −18, −19 and −20, relative to the PAM, that are the location of cytosine deamination, which leads to C-to-T mutagenesis and the introduction of a stop codon truncating the BT_0364 coding sequence. The middle Sanger trace shows the BT_0364 edited sample and the lower graph shows the C-to-T mutation frequencies. FIG. 6B discloses SEQ ID NOS 21-23, respectively, in order of appearance.

### DETAILED DESCRIPTION

The present disclosure provides engineered RNA-guided genome modifying systems that can be used to modify specific DNA sequences. In particular, the RNA-guided genome modifying systems are engineered to target specific loci in chromosomal DNA of the targeted members of domain Bacteria, specifically members of the phylum Bacteroidetes belonging to the genus Bacteroides, including those members residing in one or more body habitats of a host animal species (including but not limited to H. sapiens) resulting in the modification of genomic DNA sequences (e.g., knockout, knockin).

### (I) Protein-Nucleic Acid Complexes

One aspect of the present disclosure provides a protein-nucleic acid complex comprising an engineered RNA-guided nucleobase modifying system in association with a chromosome of a target bacterial species (or strain level variant of that species), wherein the engineered RNA-guided nucleobase modifying system is targeted to a specific locus in the chromosome of the organism, and chromosome of the organism encodes an HU family DNA-binding protein comprising an amino acid sequence having at least 50% sequence identity to the amino acid sequence of SEQ ID NO: 1: (MNKADLISAVAAEAGLSKVDAKKAVEAFVSTVTKALQEGDKVSLIGFGTFSV AERSARTGINPSTKATITIPAKKVTKFKPGAELADAIK) (e.g., at least 55%, at least 60%, at least 65%, at least 70%, at least 75%, at least 80%, at least 85%, at least 90%, at least 95%, or at least 99% sequence identity), and the chromosome of the species/strain is associated with HU family DNA-binding proteins have at least 50% sequence identity to the amino acid sequence of SEQ ID NO: 1 (e.g., at least 55%, at least 60%, at least 65%, at least 70%, at least 75%, at least 80%, at least 85%, at least 90%, at least 95%, or at least 99% sequence identity).

In various embodiments, the RNA-guided nucleobase modifying system comprises (i) a clustered regularly interspaced short palindromic repeats (CRISPR) system comprising a CRISPR protein and a guide RNA (gRNA) and (ii) a nucleobase modifying enzyme or catalytic domain thereof, wherein the CRISPR protein is a nuclease deficient CRISPR variant (e.g., dead CRISPR) or a CRISPR nickase. The gRNA of CRISPR system is engineered to direct the binding of the RNA-guided nucleobase modifying system to the specific locus in the chromosome of the bacterial species/strain. Because the CRISPR protein is, in some embodiments, a nuclease deficient CRISPR variant or a CRISPR nickase, one or more nucleobases in the specific locus of the bacterial chromosome can be modified without the generation of a double stranded break, which can be lethal, in the chromosome of the organism. The bacterial organism expresses the HU family protein, which associates with the bacterial chromosomal DNA. Thus, the protein-nucleic acid complexes disclosed herein comprise ribonucleoprotein complexes (gRNA/CRISPR protein/nucleobase modifying enzyme) bound to DNA/protein complexes (bacterial chromosomal DNA and associated HU family proteins).

(a) Engineered RNA-Guided Nucleobase Modifying Systems

The protein-nucleic acid complexes disclosed herein typically comprise engineered RNA-guided nucleobase modifying system that comprise (i) a CRISPR system comprising a CRISPR protein and a guide RNA (gRNA), wherein the CRISPR protein is a nuclease deficient CRISPR variant or a CRISPR nickase and (ii) a nucleobase modifying enzyme or catalytic domain thereof.

(i) CRISPR Systems

RNA-guided CRISPR systems are naturally-occurring defense mechanisms in bacteria and archaea that have been repurposed as RNA-guided DNA-targeting platforms used for gene editing in many cell types. See, e.g., International Publication Number WO 2014/089190 to Chen et al. (hereby incorporated by reference herein in its entirety). As detailed below, the guide RNA, which interacts with the CRISPR protein, can be engineered to base pair with a specific sequence in a nucleic acid of interest, thereby targeting the CRISPR protein to the specific sequence in the nucleic acid of interest.

The CRISPR system of the RNA-guided nucleobase modifying systems disclosed herein can be derived from a Type I CRISPR system, a type II CRISPR system, a type III CRISPR system, a Type IV CRISPR system, a type V CRISPR system, or a type VI CRISPR system. In specific embodiments, the CRISPR nuclease can be from single-subunit effector systems such as Type II, Type V, or Type VI systems. In various embodiments, the CRISPR protein can be derived from a Type II Cas9 protein, a Type V Cas12 (formerly called Cpf1) protein, a Type VI Cas13 (formerly called C2cd) protein, a CasX protein, or a CasY protein. In one particular embodiment, the CRISPR nuclease is derived from a Type II Cas9 protein. In another particular embodiment, the CRISPR nuclease is derived from a Type V Cas12 protein.

The CRISPR protein can be derived from Acaryochloris spp., Acetohalobium spp., Acidaminococcus spp., Acidithiobacillus spp., Acidothermus spp., Akkermansia spp., Alicyclobacillus spp., Allochromatium spp., Ammonifex spp., Anabaena spp., Arthrospira spp., Bacillus spp., Bifidobacterium spp., Burkholderiales spp., Caldicelulosiruptor spp., Campylobacterspp., Candidatus spp., Clostridium spp., Corynebacterium spp., Crocosphaera spp., Cyanothece spp., Deltaproteobacterium spp., Exiguobacterium spp., Finegoldia spp., Francisella spp., Ktedonobacter spp., Lachnospiraceae spp., Lactobacillus spp., Leptotrichia spp., Lyngbya spp., Marinobacter spp., Methanohalobium spp., Microscilla spp., Microcoleus spp., Microcystis spp., Mycoplasma spp., Natranaerobius spp., Neisseria spp., Nitratifractor spp., Nitrosococcus spp., Nocardiopsis spp., Nodularia spp., Nostoc spp., Oenococcus spp., Oscillatoria spp., Parasutterella spp., Pelotomaculum spp., Petrotoga spp., Planctomyces spp., Polaromonas spp., Prevotella spp., Pseudoalteromonas spp., Ralstonia spp., Ruminococcus spp., Staphylococcus spp., Streptococcus spp., Streptomyces spp., Streptosporangium spp., Synechococcus spp., Thermosipho spp., Verrucomicrobia spp., Wolinella spp., and/or species delineated in bioinformatic surveys of genomic databases such as those disclosed in Makarova, Kira S., et al. “An updated evolutionary classification of CRISPR-Cas systems.” Nature Reviews Microbiology 13.11 (2015): 722 and Koonin, Eugene V., Kira S. Makarova, and Feng Zhang. “Diversity, classification and evolution of CRISPR-Cas systems.” Current opinion in microbiology 37 (2017): 67-78, each of which is hereby incorporated by reference herein in their entirety.

In some aspects, the CRISPR protein can be derived from Streptococcus pyogenes Cas9, Francisella novicida Cas9, Staphylococcus aureus Cas9, Streptococcus thermophilus Cas9, Streptococcus pasteurianus Cas9, Campylobacter jejuni Cas9, Neisseria meningitis Cas9, Neisseria cinerea Cas9, Francisella novicida Cas12a, Acidaminococcus sp. Cas12a Lachnospiraceae bacterium ND2006 Cas12a, Leptotrichia wadeii Cas13a, Leptotrichia shahii Cas13a, Prevotella sp. P5-125 Cas13, Ruminococcus flavefaciens Cas13d, Deltaproteobacterium CasX, Planctomyces CasX, or Candidatus CasY.

In some embodiments, the CRISPR protein of the RNA-guided nucleobase modifying systems disclosed herein can be a nuclease deficient CRISPR variant, which has been modified to be devoid of all nuclease activity. Wild-type CRISPR nucleases generally comprise two nuclease domains, e.g., Cas9 nucleases comprise RuvC and HNH domains, each of which cleaves one strand of a double-stranded sequence. One or more mutations in the RuvC nuclease domain and the HNH nuclease domain can eliminate all nuclease activity. For example, nuclease deficient CRISPR variants can comprise mutations such as D10A, DBA, E762A, and/or D986A in the RuvC domain, and mutations such as H840A, H559A, N854A, N856A, and/or N863A in the HNH domain (with reference to the numbering system of Streptococcus pyogenes Cas9, SpyCas9). Nuclease deficient Cas12 variants can comprise comparable mutations in the two nuclease domains. In some embodiments, the nuclease deficient CRISPR variant can be a dead Cas9 (dCas9) variant with D10A and H840A mutations.

In other embodiments, the CRISPR protein of the RNA-guided nucleobase modifying systems disclosed herein can be a CRISPR nickase, which cleaves one strand of a double-stranded sequence. The nickase can be engineered via inactivation of one of the nuclease domains of the CRISPR nuclease. For example, the RuvC domain or the HNH domain of a Cas9 protein can be inactivated by one or more mutations as described above to generate a Cas9 nickase (e.g., nCas9). Comparable mutations in other CRISPR nucleases can generate other CRISPR nickases (e.g., nCas12).

Additionally, the CRISPR protein can be modified to have improved targeting specificity, improved fidelity, altered PAM specificity, and/or increased stability. For example, the CRISPR protein can be modified to comprise one or more mutations (i.e., substitution, deletion, and/or insertion of at least one amino acid). Non-limiting examples of mutations that improve targeting specificity, improve fidelity, and/or decrease off-target effects include N497A, R661A, Q695A, K810A, K848A, K855A, Q926A, K1003A, R1060A, and/or D1135E (with reference to the numbering system of SpyCas9).

A CRISPR system also comprises a guide RNA. A guide RNA interacts with the CRISPR protein and a target sequence in the nucleic acid of interest and guides the CRISPR protein to the target sequence. The target sequence has no sequence limitation except that the sequence is adjacent to a protospacer adjacent motif (PAM) sequence. Different CRISPR proteins recognize different PAM sequences. For example, PAM sequences for Cas9 proteins include 5′-NGG, 5′-NGGNG, 5′-NNAGAAW, 5′-NNNNGATT, 5-NNNNRYAC, 5′-NNNNCAAA, 5′-NGAAA, 5′-NNAAT, 5′-NNNRTA, 5′-NNGG, 5′-NNNRTA, 5′-MMACCA, 5′-NNNNGRY, 5′-NRGNK, 5′-GGGRG, 5′-NNAMMMC, and 5′-NNG, and PAM sequences for Cas12a proteins include 5′-TTN and 5′-TTTV, wherein N is defined as any nucleotide, R is defined as either G or A, W is defined as either A or T, Y is defined an either C or T, and V is defined as A, C, or G. In general, Cas9 PAMs are located 3′ of the target sequence, and Cas12a PAMs are located 5′ of the target sequence. Various PAM sequences and the CRISPR proteins that recognize them are known in the art, e.g., U.S. Patent Application Publication 2019/0249200; Leenay, Ryan T., et al. “Identifying and visualizing functional PAM diversity across CRISPR-Cas systems.” Molecular cell 62.1 (2016): 137-147; and Kleinstiver, Benjamin P., et al. “Engineered CRISPR-Cas9 nucleases with altered PAM specificities.” Nature 523.7561 (2015): 481, each of which are incorporated by reference herein in their entirety

Guide RNAs are engineered to complex with specific CRISPR proteins. In general, a guide RNA comprises (i) a CRISPR RNA (crRNA) that comprises a guide or spacer sequence at the 5′ end that hybridizes at the target site, and (ii) a transacting crRNA (tracrRNA) sequence that interacts with the crRNA and the CRISPR protein. The guide or spacer sequence of each guide RNA is different (i.e., is sequence specific). The rest of the guide RNA sequence is generally the same in guide RNAs designed to complex with a specific CRISPR protein.

The crRNA comprises the guide sequence at the 5′ end, as well as additional sequence at the 3′ end that base-pairs with sequence at the 5′ end of the tracrRNA to form a duplex structure, and the tracrRNA comprises additional sequence that forms at least one stem-loop structure, which interacts with the CRISPR nuclease. The guide RNA can be a single molecule (e.g., a single guide RNA (sgRNA) or 1-piece sgRNA), wherein the crRNA sequence is linked to the tracrRNA sequence. Alternatively, the guide RNA can be a dual molecule gRNA comprising separate molecules, i.e., crRNA and tracrRNA.

The crRNA guide sequence is designed to hybridize with the complement of a target sequence (i.e., protospacer) in the nucleic acid of interest. The “target nucleic acid” is a double-stranded molecule; one strand comprises the target sequence and is referred to as the “PAM strand,” and the other complementary strand is referred to as the “non-PAM strand.” One of skill in the art recognizes that the gRNA spacer sequence hybridizes to the reverse complement of the target sequence, which is located in the non-PAM strand of the target nucleic acid. In general, the sequence identity between the guide sequence and the target sequence is at least 80%, at least 85%, at least 90%, at least 95%, or at least 99%. In specific embodiments, the complementarity is complete (i.e., 100%). In various embodiments, the length of the crRNA guide sequence can range from about 15 nucleotides to about 25 nucleotides. For example, the crRNA guide sequence can be about 15, 16, 17, 18, 19, 20, 21, 22, 23, 24, or 25 nucleotides in length. In specific embodiments, the guide is about 19, 20, or 21 nucleotides in length. In one embodiment, the crRNA guide sequence has a length of 20 nucleotides. In certain embodiments, the crRNA can comprise additional 3′ sequence that interacts with tracrRNA. The additional sequence can comprise from about 10 to about 40 nucleotides. In embodiments in which the guide RNA comprises a single molecule, the crRNA and tracrRNA portions of the gRNA can be linked by sequence that forms a loop. The sequence that form the loop can range in length from about 4 nucleotides to about 10 or more nucleotides.

As mentioned above, the tracrRNA comprises repeat sequences that form at least one stem loop structure, which interacts with the CRISPR nuclease. The length of each loop and stem can vary. For example, the loop can range from about 3 to about 10 nucleotides in length, and the stem can range from about 6 to about 20 base pairs in length. The stem can comprise one or more bulges of 1 to about 10 nucleotides. The tracrRNA sequence in the guide RNA generally is based upon the sequence of wild type tracrRNA that interact with the wild-type CRISPR nuclease. The wild-type sequence can be modified to facilitate secondary structure formation, increased secondary structure stability, and the like. For example, one or more nucleotide changes can be introduced into the guide RNA sequence. The tracrRNA sequence can range in length from about 50 nucleotides to about 300 nucleotides. In various embodiments, the tracrRNA can range in length from about 50 to about 90 nucleotides, from about 90 to about 110 nucleotides, from about 110 to about 130 nucleotides, from about 130 to about 150 nucleotides, from about 150 to about 170 nucleotides, from about 170 to about 200 nucleotides, from about 200 to about 250 nucleotides, or from about 250 to about 300 nucleotides. The tracrRNA can comprise an optional extension at the 3′ end of the tracrRNA.

The guide RNA can comprise standard ribonucleotides and/or modified ribonucleotides. In some embodiments, the guide RNA can comprise standard or modified deoxyribonucleotides. In embodiments in which the guide RNA is enzymatically synthesized (i.e., in vivo or in vitro), the guide RNA generally comprises standard ribonucleotides. In embodiments in which the guide RNA is chemically synthesized, the guide RNA can comprise standard or modified ribonucleotides and/or deoxyribonucleotides. Modified ribonucleotides and/or deoxyribonucleotides include base modifications (e.g., pseudouridine, 2-thiouridine, N6-methyladenosine, and the like) and/or sugar modifications (e.g., 2′-O-methy, 2′-fluoro, 2′-amino, locked nucleic acid (LNA), and so forth). The backbone of the guide RNA can also be modified to comprise phosphorothioate linkages, boranophosphate linkages, or peptide nucleic acids.

Optional Aptamer Sequence.

In some situations, the CRISPR protein or the tracrRNA of the guide RNA can further comprise one or more aptamer sequences (Konermann et al., Nature, 2015, 517(7536):583-588; Zalatan et al., Cell, 2015, 160(1-2):339-50). The aptamer sequence can be nucleic acid (e.g., RNA) or peptide. Aptamer sequence can be recognized and bound by specific adaptor proteins. Non-limiting examples of suitable aptamer sequences include MS2/MSP, PP7/PCP, Com, N22, AP205, BZ13, F1, F2, fd, fr, GA, ID2, JP34, JP500, JP501, KU1, M11, M12, MX1, NL95, PRR1, ϕCb5, ϕCb8r, ϕCb12r, ϕCb23r, Qβ, R17, SP, TW18, TW19, VK, and 7s. Those of skill in the art appreciate that the length of the aptamer sequence can vary. The aptamer sequence can be linked directly to the CRISPR protein or the tracrRNA via a covalent bond. Alternatively, the aptamer sequence can be linked indirectly to the CRISPR protein or the tracrRNA via a linker.

Linkers are chemical groups that connect one or more other chemical groups via at least one covalent bond. Suitable linkers include amino acids, peptides, nucleotides, nucleic acids, organic linker molecules (e.g., maleimide derivatives, N-ethoxybenzylimidazole, biphenyl-3,4′,5-tricarboxylic acid, p-aminobenzyloxycarbonyl, and the like), disulfide linkers, and polymer linkers (e.g., PEG). The linker can include one or more spacing groups including, but not limited to alkylene, alkenylene, alkynylene, alkyl, alkenyl, alkynyl, alkoxy, aryl, heteroaryl, aralkyl, aralkenyl, aralkynyl and the like. The linker can be neutral, or carry a positive or negative charge. In some embodiments, the linker can be a peptide linker. The peptide linker can be a flexible amino acid linker (e.g., comprising small, non-polar or polar amino acids). Alternatively, the peptide linker can be a rigid amino acid linker (e.g., α-helical). Peptide likers can vary in length from about four amino acids up to a hundred or more amino acids. For example, suitable linkers can comprise 10-20 amino acids, 20-40 amino acids, 40-80 amino acids, or 80-120 amino acids. Examples of suitable linkers are well known in the art and programs to design linkers are readily available (Crasto et al., Protein Eng., 2000, 13(5):309-312).

(ii) Nucleobase Modifying Enzymes

The engineered RNA-guided (CRISPR) nucleobase modifying systems disclosed herein also comprise a nucleobase modifying enzyme or catalytic domain thereof.

A variety of nucleobase modifying enzymes are suitable for use on the systems disclosed herein. The nucleobase modifying enzyme can be a DNA base editor. In some embodiments, the DNA base editor can be a cytidine deaminase, which converts cytidine into uridine, which is read by polymerase enzymes as thymine. Non-limiting examples of cytidine deaminases include cytidine deaminase 1 (CDA1), cytidine deaminase 2 (CDA2), activation-induced cytidine deaminase (AICDA), apolipoprotein B mRNA-editing complex (APOBEC) family cytidine deaminase (e.g., APOBEC1, APOBEC2, APOBEC3A, APOBEC3B, APOBEC3C, APOBEC3D/E, APOBEC3F, APOBEC3G, APOBEC3H, APOBEC4), APOBEC1 complementation factor/APOBEC1 stimulating factor (ACF1/ASF) cytidine deaminase, cytosine deaminase acting on RNA (CDAR), bacterial long isoform cytidine deaminase (CDDL), and cytosine deaminase acting on tRNA (CDAT). In other embodiments, the DNA base editor can be an adenosine deaminase, which converts adenosine into inosine, which is read by polymerase enzymes as guanosine. Non-limiting examples of adenosine deaminases include tRNA adenine deaminase, adenosine deaminase, adenosine deaminase acting on RNA (ADAR), and adenosine deaminase acting on tRNA (ADAT).

The nucleobase modifying enzyme (base editor) can be wild type or a fragment thereof, a modified version thereof (e.g., non-essential domains can be deleted), or an engineered version thereof. The nucleobase modifying enzyme (base editor) can be of eukaryotic, bacterial, or archael origin.

In some embodiments, the nucleobase modifying enzyme (base editor) can be a cytidine deaminase or catalytic domain thereof. The cytidine deaminase can be of human, mouse, lamprey, abalone, or E. coli origin. In embodiments in which the nucleobase modifying enzyme is a cytidine deaminase, the RNA-guided nucleobase modifying system can further comprise at least one uracil glycosylase inhibitor (UGI) domain. Removal of uracil from DNA, which is the result of cytosine deamination, is inhibited by UGI. Suitable UGI domains are known in the art.

In some embodiments, a system that employs a cytidine deaminase and a UGI may have negative effects if these components are overexpressed. To prevent overexpression, a degradation tag may be added. Degradation tags signal a protein to be degraded by the protein recycling system. These degradation tags result in different protein half-lives. Non-limiting degradation tag examples are LVA, AAV, ASV and LAA.

Optional Adaptor Protein.

In some embodiments, the nucleobase modifying enzyme or catalytic domain thereof can be linked to an adaptor protein that recognizes and binds an aptamer sequence. In some embodiments, the adaptor protein can be MS2 bacteriophage coat protein that recognizes and binds MCP aptamer sequence or PP7 bacteriophage coat protein that recognizes and binds PCP aptamer sequence. In other embodiments, the adaptor protein can recognize and bind Com, N22, AP205, BZ13, F1, F2, fd, fr, GA, ID2, JP34, JP500, JP501, KU1, M11, M12, MX1, NL95, PRR1, ϕCb5, ϕCb8r, ϕCb12r, ϕCb23r, Qβ, R17, SP, TW18, TW19, VK, or 7s adaptor sequences.

The linkage between the nucleobase modifying enzyme or catalytic domain thereof and the adaptor protein can be direct via a covalent bond. Alternatively, the linkage between the nucleobase modifying enzyme or catalytic domain thereof and the adaptor protein can be indirect via a linker. Linkers are described above in section (I)(a)(i). The adaptor protein can be linked to the amino terminus and/or the carboxy terminus of the nucleobase modifying enzyme or catalytic domain thereof.

(iii) Interactions Between CRISPR System and Nucleobase Modifying Enzyme

The engineered RNA-guided nucleobase modifying systems disclosed herein comprise (i) a CRISPR system having no nuclease activity or having nickase activity (described above in section (I)(a)(i)) and (ii) a nucleobase modifying enzyme (base editor) or catalytic domain thereof (described above in section (I)(a)(ii)). The CRISPR system and the nucleobase modifying enzyme or catalytic domain thereof can interact in a variety of ways.

In some embodiments, the CRISPR protein of the CRISPR system can be linked to the nucleobase modifying enzyme or catalytic domain thereof. In some aspects, the linkage between the CRISPR protein and the nucleobase modifying enzyme or catalytic domain thereof can be direct via a covalent bond (e.g., peptide bond). In other aspects, the linkage between the CRISPR protein and the nucleobase modifying enzyme or catalytic domain thereof can be via a linker. Linkers are described above in section (I)(a)(i). The nucleobase modifying enzyme or catalytic domain thereof can be linked to the amino terminus and/or the carboxy terminus of the CRISPR protein.

In other embodiments, the nucleobase modifying enzyme or catalytic domain thereof can be linked to an adaptor protein (described above in section (I)(a)(ii)) and the CRISPR protein or the gRNA can comprise an aptamer sequence (described above in section (I)(a)(i)) capable of binding the adaptor protein. For example, the nucleobase modifying enzyme (e.g., cytidine/adenosine deaminase) can be linked to a MS2 bacteriophage coat protein, and the gRNA of the CRISPR system can comprise an MCP aptamer sequence that forms a stem-loop structure, wherein the MS2 protein can bind the MSP aptamer sequence thereby forming a CRISPR-cytidine/adenosine deaminase system.

(iv) Expression of Engineered RNA-Guided Nucleobase Modifying Systems

The guide RNA of the CRISPR system is engineered to target the RNA-guided (CRISPR) nucleobase modifying system to a specific locus in bacterial chromosomal DNA such that the protein-nucleic acid complexes, as described above, can be formed. In general, the protein-nucleic acid complex is formed within the bacterial cell.

In some embodiments, the engineered RNA-guided (CRISPR) nucleobase modifying system can be expressed from at least one nucleic acid encoding said system that is integrated into the chromosome of the bacterial species or strain. In other embodiments, the engineered RNA-guided (CRISPR) nucleobase modifying system can be expressed from at least one nucleic acid encoding said system that is carried on at least one extrachromosomal vector. Techniques for introducing nucleic acids into bacteria are well known in the art, as are means for integrating nucleic acids into the bacterial chromosome.

Expression of the engineered RNA-guided (CRISPR) nucleobase modifying system can be regulated. For example, the expression of the engineered CRISPR nuclease system can be regulated by an inducible promoter, as described below in section (II).

In some embodiments, the engineered RNA-guided (CRISPR) nucleobase modifying system can be formatted as a pooled guide RNA library to target many genome locations in parallel, enabling the creation of a population of Bacteroides cells, each cell having a different RNA-guided genome modification. These pooled cell populations may then be placed under selective pressure, and the selected cells analyzed by DNA sequencing.

(b) Bacterial Chromosome

The protein-nucleic acid complex disclosed herein further comprises a bacterial chromosome, wherein the bacterial chromosome encodes HU family DNA-binding protein comprising an amino acid sequence with at least 50% sequence identity to the amino acid sequence of SEQ ID NO: 1 (at least 55%, at least 60%, at least 65%, at least 70%, at least 75%, at least 80%, at least 85%, at least 90%, at least 95%, or at least 99% sequence identity to SEQ ID NO: 1), and the chromosomal DNA of the bacterium is associated with said HU family DNA-binding protein. The HU family of DNA-binding proteins comprises small (˜90 amino acids) basic histone-like proteins that bind double stranded DNA without sequence specificity and bind DNA structures such as forks, three/four way junctions, nicks, overhangs, and bulges. Binding of HU family DNA-binding proteins can stabilize the DNA and protect it from denaturation under extreme environmental conditions. The association of Bacteroides HU family DNA proteins with chromosomal DNA creates a unique structural environment with which other DNA binding proteins, such as those of CRISPR systems, must be compatible in order to bind chromosomal targets and function as nucleases, nickases, deaminases, or other genome modification modalities.

In general, the chromosome (or chromosomal region thereof) can be within any member of Bacteroidetes. In some embodiments, the HU family DNA-binding protein comprises an amino acid sequence having at least 50%, at least 55%, at least 60%, at least 65%, at least 70%, at least 75%, at least 80%, at least 85%, at least 90%, at least 95%, or at least 99% sequence identity to SEQ ID NO: 1. In other embodiments, the HU family DNA-binding protein has the amino acid sequence of SEQ ID NO: 1.

In some embodiments, the organism is a member of the genus Bacteroides. Bacteroides species are prominent anaerobic symbionts of mammalian gut microbiota. They contain a variety of saccharolytic enzymes and are the primary fermenters of polysaccharides in the gut. They maintain complex and generally beneficial relationships with the host when retained in the gut, but can cause significant pathology if they escape this environment. Non-limiting examples of Bacteroides species include B. acidifaciens, B. bacterium, B. barnesiaes, B. caccae, B. caecicola, B. caecigallinarum, B. capillosis, B. cellulosilyticus, B. cellulosolvens, B. clarus, B. coagulans, B. coprocola, B. coprophilus, B. coprosuis, B. distasonis, B. dorei, B. eggerthii, B. gracilis, B. faecichinchillae, B. faecis, B. finegoldii, B. fluxus, B. fragilis, B. galacturonicus, B. gallinaceum, B. gallinarum, B. goldsteinii, B. graminisolvens, B. helcogene, B. heparinolyticus, B. intestinalis, B. johnsonii, B. luti, B. massiliensis, B. melaninogenicus, B. neonati, B. nordii, B. oleiciplenus, B. oris, B. ovatus, B. paurosaccharolyticus, B. plebeius, B. polypragmatus, B. pro pionicifaciens, B. putredinis, B. pyogenes, B. reticulotermitis, B. rodentium, B. salanitronis, B. salyersiae, B. sartorii, B. sediment, B. stercoris, B. stercorirosoris, B. suis, B. tectus, B. thetaiotaomicron, B. timonensis, B. uniformis, B. vulgatus, B. xylanisolvens, B. xylanolyticus, and B. zoogleoformans and strain level variants of these species. For example, strain level variants of B. cellulosilyticus include, but are not limited to, B. cellulosilyticus DSM 14838, B. cellulosilyticus WH2, B. cellulosilyticus CL02T12C19, B. cellulosilyticus CRE21(T), and B. cellulosilyticus JCM 15632T.

In some embodiments, the chromosome (or chromosomal region thereof) is chosen from Bacteroides thetaiotaomicron, Bacteroides vulgatus, Bacteroides cellulosilyticus, Bacteroides fragilis, Bacteroides helcogenes, Bacteroides ovatus, Bacteroides salanitronis, Bacteroides uniformis, or Bacteroides xylanisolvens and strain level variants of these species.

In some embodiments, the chromosome (or chromosomal region thereof) is chosen from Bamesiella sp., Bamesiella viscericola, Capnocytphaga sp., Odoribacter splanchnicus, Paludibacter sp., Parabacteroides sp., Porphyromonadaceae bacterium, and Schleiferia sp. and strain level variants of these species.

The chromosomal region, for example, can be of length associated with plasmid DNA or bacterial artificial chromosomes (approximately 2,000 to 350,000 bases in length) or of lengths associated with primary bacterial chromosomes (130,000 bases to 14,000,000 bases in length).

Thus, for example, the length of the chromosomal region can be about 2000, about 3000, about 4000, about 5000, about 6000, about 7000, about 8000, about 9000, about 10000, about 11000, about 12000, about 13000, about 14000, about 15000, about 16000, about 17000, about 18000, about 19000, about 20000, about 21000, about 22000, about 23000, about 24000, about 25000, about 26000, about 27000, about 28000, about 29000, about 30000, about 31000, about 32000, about 33000, about 34000, about 35000, about 36000, about 37000, about 38000, about 39000, about 40000, about 41000, about 42000, about 43000, about 44000, about 45000, about 46000, about 47000, about 48000, about 49000, about 50000, about 51000, about 52000, about 53000, about 54000, about 55000, about 56000, about 57000, about 58000, about 59000, about 60000, about 61000, about 62000, about 63000, about 64000, about 65000, about 66000, about 67000, about 68000, about 69000, about 70000, about 71000, about 72000, about 73000, about 74000, about 75000, about 76000, about 77000, about 78000, about 79000, about 80000, about 81000, about 82000, about 83000, about 84000, about 85000, about 86000, about 87000, about 88000, about 89000, about 90000, about 91000, about 92000, about 93000, about 94000, about 95000, about 96000, about 97000, about 98000, about 99000, about 100000, about 101000, about 102000, about 103000, about 104000, about 105000, about 106000, about 107000, about 108000, about 109000, about 110000, about 111000, about 112000, about 113000, about 114000, about 115000, about 116000, about 117000, about 118000, about 119000, about 120000, about 121000, about 122000, about 123000, about 124000, about 125000, about 126000, about 127000, about 128000, about 129000, about 130000, about 131000, about 132000, about 133000, about 134000, about 135000, about 136000, about 137000, about 138000, about 139000, about 140000, about 141000, about 142000, about 143000, about 144000, about 145000, about 146000, about 147000, about 148000, about 149000, about 150000, about 151000, about 152000, about 153000, about 154000, about 155000, about 156000, about 157000, about 158000, about 159000, about 160000, about 161000, about 162000, about 163000, about 164000, about 165000, about 166000, about 167000, about 168000, about 169000, about 170000, about 171000, about 172000, about 173000, about 174000, about 175000, about 176000, about 177000, about 178000, about 179000, about 180000, about 181000, about 182000, about 183000, about 184000, about 185000, about 186000, about 187000, about 188000, about 189000, about 190000, about 191000, about 192000, about 193000, about 194000, about 195000, about 196000, about 197000, about 198000, about 199000, about 200000, about 201000, about 202000, about 203000, about 204000, about 205000, about 206000, about 207000, about 208000, about 209000, about 210000, about 211000, about 212000, about 213000, about 214000, about 215000, about 216000, about 217000, about 218000, about 219000, about 220000, about 221000, about 222000, about 223000, about 224000, about 225000, about 226000, about 227000, about 228000, about 229000, about 230000, about 231000, about 232000, about 233000, about 234000, about 235000, about 236000, about 237000, about 238000, about 239000, about 240000, about 241000, about 242000, about 243000, about 244000, about 245000, about 246000, about 247000, about 248000, about 249000, about 250000, about 251000, about 252000, about 253000, about 254000, about 255000, about 256000, about 257000, about 258000, about 259000, about 260000, about 261000, about 262000, about 263000, about 264000, about 265000, about 266000, about 267000, about 268000, about 269000, about 270000, about 271000, about 272000, about 273000, about 274000, about 275000, about 276000, about 277000, about 278000, about 279000, about 280000, about 281000, about 282000, about 283000, about 284000, about 285000, about 286000, about 287000, about 288000, about 289000, about 290000, about 291000, about 292000, about 293000, about 294000, about 295000, about 296000, about 297000, about 298000, about 299000, about 300000, about 301000, about 302000, about 303000, about 304000, about 305000, about 306000, about 307000, about 308000, about 309000, about 310000, about 311000, about 312000, about 313000, about 314000, about 315000, about 316000, about 317000, about 318000, about 319000, about 320000, about 321000, about 322000, about 323000, about 324000, about 325000, about 326000, about 327000, about 328000, about 329000, about 330000, about 331000, about 332000, about 333000, about 334000, about 335000, about 336000, about 337000, about 338000, about 339000, about 340000, about 341000, about 342000, about 343000, about 344000, about 345000, about 346000, about 347000, about 348000, about 349000, about 350000, about 351000, about 352000, about 353000, about 354000, about 355000, about 356000, about 357000, about 358000, about 359000, about 360000, about 361000, about 362000, about 363000, about 364000, about 365000, about 366000, about 367000, about 368000, about 369000, about 370000, about 371000, about 372000, about 373000, about 374000, about 375000, about 376000, about 377000, about 378000, about 379000, about 380000, about 381000, about 382000, about 383000, about 384000, about 385000, about 386000, about 387000, about 388000, about 389000, about 390000, about 391000, about 392000, about 393000, about 394000, about 395000, about 396000, about 397000, about 398000, about 399000, about 400000, about 401000, about 402000, about 403000, about 404000, about 405000, about 406000, about 407000, about 408000, about 409000, about 410000, about 411000, about 412000, about 413000, about 414000, about 415000, about 416000, about 417000, about 418000, about 419000, about 420000, about 421000, about 422000, about 423000, about 424000, about 425000, about 426000, about 427000, about 428000, about 429000, about 430000, about 431000, about 432000, about 433000, about 434000, about 435000, about 436000, about 437000, about 438000, about 439000, about 440000, about 441000, about 442000, about 443000, about 444000, about 445000, about 446000, about 447000, about 448000, about 449000, about 450000, about 451000, about 452000, about 453000, about 454000, about 455000, about 456000, about 457000, about 458000, about 459000, about 460000, about 461000, about 462000, about 463000, about 464000, about 465000, about 466000, about 467000, about 468000, about 469000, about 470000, about 471000, about 472000, about 473000, about 474000, about 475000, about 476000, about 477000, about 478000, about 479000, about 480000, about 481000, about 482000, about 483000, about 484000, about 485000, about 486000, about 487000, about 488000, about 489000, about 490000, about 491000, about 492000, about 493000, about 494000, about 495000, about 496000, about 497000, about 498000, about 499000, about 500000, about 501000, about 502000, about 503000, about 504000, about 505000, about 506000, about 507000, about 508000, about 509000, about 510000, about 511000, about 512000, about 513000, about 514000, about 515000, about 516000, about 517000, about 518000, about 519000, about 520000, about 521000, about 522000, about 523000, about 524000, about 525000, about 526000, about 527000, about 528000, about 529000, about 530000, about 531000, about 532000, about 533000, about 534000, about 535000, about 536000, about 537000, about 538000, about 539000, about 540000, about 541000, about 542000, about 543000, about 544000, about 545000, about 546000, about 547000, about 548000, about 549000, about 550000, about 551000, about 552000, about 553000, about 554000, about 555000, about 556000, about 557000, about 558000, about 559000, about 560000, about 561000, about 562000, about 563000, about 564000, about 565000, about 566000, about 567000, about 568000, about 569000, about 570000, about 571000, about 572000, about 573000, about 574000, about 575000, about 576000, about 577000, about 578000, about 579000, about 580000, about 581000, about 582000, about 583000, about 584000, about 585000, about 586000, about 587000, about 588000, about 589000, about 590000, about 591000, about 592000, about 593000, about 594000, about 595000, about 596000, about 597000, about 598000, about 599000, about 600000, about 601000, about 602000, about 603000, about 604000, about 605000, about 606000, about 607000, about 608000, about 609000, about 610000, about 611000, about 612000, about 613000, about 614000, about 615000, about 616000, about 617000, about 618000, about 619000, about 620000, about 621000, about 622000, about 623000, about 624000, about 625000, about 626000, about 627000, about 628000, about 629000, about 630000, about 631000, about 632000, about 633000, about 634000, about 635000, about 636000, about 637000, about 638000, about 639000, about 640000, about 641000, about 642000, about 643000, about 644000, about 645000, about 646000, about 647000, about 648000, about 649000, about 650000, about 651000, about 652000, about 653000, about 654000, about 655000, about 656000, about 657000, about 658000, about 659000, about 660000, about 661000, about 662000, about 663000, about 664000, about 665000, about 666000, about 667000, about 668000, about 669000, about 670000, about 671000, about 672000, about 673000, about 674000, about 675000, about 676000, about 677000, about 678000, about 679000, about 680000, about 681000, about 682000, about 683000, about 684000, about 685000, about 686000, about 687000, about 688000, about 689000, about 690000, about 691000, about 692000, about 693000, about 694000, about 695000, about 696000, about 697000, about 698000, about 699000, about 700000, about 701000, about 702000, about 703000, about 704000, about 705000, about 706000, about 707000, about 708000, about 709000, about 710000, about 711000, about 712000, about 713000, about 714000, about 715000, about 716000, about 717000, about 718000, about 719000, about 720000, about 721000, about 722000, about 723000, about 724000, about 725000, about 726000, about 727000, about 728000, about 729000, about 730000, about 731000, about 732000, about 733000, about 734000, about 735000, about 736000, about 737000, about 738000, about 739000, about 740000, about 741000, about 742000, about 743000, about 744000, about 745000, about 746000, about 747000, about 748000, about 749000, about 750000, about 751000, about 752000, about 753000, about 754000, about 755000, about 756000, about 757000, about 758000, about 759000, about 760000, about 761000, about 762000, about 763000, about 764000, about 765000, about 766000, about 767000, about 768000, about 769000, about 770000, about 771000, about 772000, about 773000, about 774000, about 775000, about 776000, about 777000, about 778000, about 779000, about 780000, about 781000, about 782000, about 783000, about 784000, about 785000, about 786000, about 787000, about 788000, about 789000, about 790000, about 791000, about 792000, about 793000, about 794000, about 795000, about 796000, about 797000, about 798000, about 799000, about 800000, about 801000, about 802000, about 803000, about 804000, about 805000, about 806000, about 807000, about 808000, about 809000, about 810000, about 811000, about 812000, about 813000, about 814000, about 815000, about 816000, about 817000, about 818000, about 819000, about 820000, about 821000, about 822000, about 823000, about 824000, about 825000, about 826000, about 827000, about 828000, about 829000, about 830000, about 831000, about 832000, about 833000, about 834000, about 835000, about 836000, about 837000, about 838000, about 839000, about 840000, about 841000, about 842000, about 843000, about 844000, about 845000, about 846000, about 847000, about 848000, about 849000, about 850000, about 851000, about 852000, about 853000, about 854000, about 855000, about 856000, about 857000, about 858000, about 859000, about 860000, about 861000, about 862000, about 863000, about 864000, about 865000, about 866000, about 867000, about 868000, about 869000, about 870000, about 871000, about 872000, about 873000, about 874000, about 875000, about 876000, about 877000, about 878000, about 879000, about 880000, about 881000, about 882000, about 883000, about 884000, about 885000, about 886000, about 887000, about 888000, about 889000, about 890000, about 891000, about 892000, about 893000, about 894000, about 895000, about 896000, about 897000, about 898000, about 899000, about 900000, about 901000, about 902000, about 903000, about 904000, about 905000, about 906000, about 907000, about 908000, about 909000, about 910000, about 911000, about 912000, about 913000, about 914000, about 915000, about 916000, about 917000, about 918000, about 919000, about 920000, about 921000, about 922000, about 923000, about 924000, about 925000, about 926000, about 927000, about 928000, about 929000, about 930000, about 931000, about 932000, about 933000, about 934000, about 935000, about 936000, about 937000, about 938000, about 939000, about 940000, about 941000, about 942000, about 943000, about 944000, about 945000, about 946000, about 947000, about 948000, about 949000, about 950000, about 951000, about 952000, about 953000, about 954000, about 955000, about 956000, about 957000, about 958000, about 959000, about 960000, about 961000, about 962000, about 963000, about 964000, about 965000, about 966000, about 967000, about 968000, about 969000, about 970000, about 971000, about 972000, about 973000, about 974000, about 975000, about 976000, about 977000, about 978000, about 979000, about 980000, about 981000, about 982000, about 983000, about 984000, about 985000, about 986000, about 987000, about 988000, about 989000, about 990000, about 991000, about 992000, about 993000, about 994000, about 995000, about 996000, about 997000, about 998000, about 999000, about 1000000, about 1001000, about 1002000, about 1003000, about 1004000, about 1005000, about 1006000, about 1007000, about 1008000, about 1009000, about 1010000, about 1011000, about 1012000, about 1013000, about 1014000, about 1015000, about 1016000, about 1017000, about 1018000, about 1019000, about 1020000, about 1021000, about 1022000, about 1023000, about 1024000, about 1025000, about 1026000, about 1027000, about 1028000, about 1029000, about 1030000, about 1031000, about 1032000, about 1033000, about 1034000, about 1035000, about 1036000, about 1037000, about 1038000, about 1039000, about 1040000, about 1041000, about 1042000, about 1043000, about 1044000, about 1045000, about 1046000, about 1047000, about 1048000, about 1049000, about 1050000, about 1051000, about 1052000, about 1053000, about 1054000, about 1055000, about 1056000, about 1057000, about 1058000, about 1059000, about 1060000, about 1061000, about 1062000, about 1063000, about 1064000, about 1065000, about 1066000, about 1067000, about 1068000, about 1069000, about 1070000, about 1071000, about 1072000, about 1073000, about 1074000, about 1075000, about 1076000, about 1077000, about 1078000, about 1079000, about 1080000, about 1081000, about 1082000, about 1083000, about 1084000, about 1085000, about 1086000, about 1087000, about 1088000, about 1089000, about 1090000, about 1091000, about 1092000, about 1093000, about 1094000, about 1095000, about 1096000, about 1097000, about 1098000, about 1099000, about 1100000, about 1101000, about 1102000, about 1103000, about 1104000, about 1105000, about 1106000, about 1107000, about 1108000, about 1109000, about 1110000, about 1111000, about 1112000, about 1113000, about 1114000, about 1115000, about 1116000, about 1117000, about 1118000, about 1119000, about 1120000, about 1121000, about 1122000, about 1123000, about 1124000, about 1125000, about 1126000, about 1127000, about 1128000, about 1129000, about 1130000, about 1131000, about 1132000, about 1133000, about 1134000, about 1135000, about 1136000, about 1137000, about 1138000, about 1139000, about 1140000, about 1141000, about 1142000, about 1143000, about 1144000, about 1145000, about 1146000, about 1147000, about 1148000, about 1149000, about 1150000, about 1151000, about 1152000, about 1153000, about 1154000, about 1155000, about 1156000, about 1157000, about 1158000, about 1159000, about 1160000, about 1161000, about 1162000, about 1163000, about 1164000, about 1165000, about 1166000, about 1167000, about 1168000, about 1169000, about 1170000, about 1171000, about 1172000, about 1173000, about 1174000, about 1175000, about 1176000, about 1177000, about 1178000, about 1179000, about 1180000, about 1181000, about 1182000, about 1183000, about 1184000, about 1185000, about 1186000, about 1187000, about 1188000, about 1189000, about 1190000, about 1191000, about 1192000, about 1193000, about 1194000, about 1195000, about 1196000, about 1197000, about 1198000, about 1199000, about 1200000, about 1201000, about 1202000, about 1203000, about 1204000, about 1205000, about 1206000, about 1207000, about 1208000, about 1209000, about 1210000, about 1211000, about 1212000, about 1213000, about 1214000, about 1215000, about 1216000, about 1217000, about 1218000, about 1219000, about 1220000, about 1221000, about 1222000, about 1223000, about 1224000, about 1225000, about 1226000, about 1227000, about 1228000, about 1229000, about 1230000, about 1231000, about 1232000, about 1233000, about 1234000, about 1235000, about 1236000, about 1237000, about 1238000, about 1239000, about 1240000, about 1241000, about 1242000, about 1243000, about 1244000, about 1245000, about 1246000, about 1247000, about 1248000, about 1249000, about 1250000, about 1251000, about 1252000, about 1253000, about 1254000, about 1255000, about 1256000, about 1257000, about 1258000, about 1259000, about 1260000, about 1261000, about 1262000, about 1263000, about 1264000, about 1265000, about 1266000, about 1267000, about 1268000, about 1269000, about 1270000, about 1271000, about 1272000, about 1273000, about 1274000, about 1275000, about 1276000, about 1277000, about 1278000, about 1279000, about 1280000, about 1281000, about 1282000, about 1283000, about 1284000, about 1285000, about 1286000, about 1287000, about 1288000, about 1289000, about 1290000, about 1291000, about 1292000, about 1293000, about 1294000, about 1295000, about 1296000, about 1297000, about 1298000, about 1299000, about 1300000, about 1301000, about 1302000, about 1303000, about 1304000, about 1305000, about 1306000, about 1307000, about 1308000, about 1309000, about 1310000, about 1311000, about 1312000, about 1313000, about 1314000, about 1315000, about 1316000, about 1317000, about 1318000, about 1319000, about 1320000, about 1321000, about 1322000, about 1323000, about 1324000, about 1325000, about 1326000, about 1327000, about 1328000, about 1329000, about 1330000, about 1331000, about 1332000, about 1333000, about 1334000, about 1335000, about 1336000, about 1337000, about 1338000, about 1339000, about 1340000, about 1341000, about 1342000, about 1343000, about 1344000, about 1345000, about 1346000, about 1347000, about 1348000, about 1349000, about 1350000, about 1351000, about 1352000, about 1353000, about 1354000, about 1355000, about 1356000, about 1357000, about 1358000, about 1359000, about 1360000, about 1361000, about 1362000, about 1363000, about 1364000, about 1365000, about 1366000, about 1367000, about 1368000, about 1369000, about 1370000, about 1371000, about 1372000, about 1373000, about 1374000, about 1375000, about 1376000, about 1377000, about 1378000, about 1379000, about 1380000, about 1381000, about 1382000, about 1383000, about 1384000, about 1385000, about 1386000, about 1387000, about 1388000, about 1389000, about 1390000, about 1391000, about 1392000, about 1393000, about 1394000, about 1395000, about 1396000, about 1397000, about 1398000, about 1399000, or about 1400000 base pairs.

(c) Specific Protein-Nucleic Acid Complexes

In specific embodiments, the protein-nucleic acid complex can comprise an engineered RNA-guided (CRISPR) nucleobase modifying system comprising (i) a nuclease deficient Cas9 or Cas12a variant and (ii) a base editor such as cytidine deaminase or adenosine deaminase (or catalytic domain thereof) bound to or associated with a Bacteroides chromosome. In some embodiments, the engineered RNA-guided (CRISPR) nucleobase modifying system comprises a nuclease deficient Cas9 or Cas12a variant linked to cytidine deaminase or adenosine deaminase (or catalytic domain thereof).

### (II) Methods for Generating the Protein-Nucleic Acid Complexes

A further aspect of the present disclosure provides methods for generating complexes comprising an engineered RNA-guided (CRISPR) nucleobase modifying system and a bacterial chromosome encoding a HU family DNA-binding protein as described above in section (I). Said methods comprise (a) engineering the CRISPR system of the nucleobase modifying system to target a specific locus in the bacterial chromosome, and (b) introducing the engineered RNA-guided (CRISPR) nucleobase modifying system into Bacteroides species/strains.

Engineering the CRISPR system of the nucleobase modifying system comprises designing a guide RNA whose crRNA guide sequence targets a specific (˜19-22 nt) sequence or locus in the bacterial chromosome that is adjacent to a PAM sequence (which is recognized by the CRISPR protein of interest) and whose tracrRNA sequence is recognized by the CRISPR protein of interest, as described above in section (I)(a)(i).

The engineered CRISPR nucleobase modifying system can be introduced into the bacterial cell as at least one encoding nucleic acid. For example, the encoding nucleic acid(s) can be part of one or more vectors. Vectors encoding the engineered CRISPR nucleobase modifying system (e.g., CRISPR-base editor fusion and one or more gRNA) can be plasmid vectors, phagemid vectors, viral vectors, bacteriophage vectors, bacteriophage-plasmid hybrid vectors, or other suitable vectors. The vector can be an integrative vector, a conjugation vector, a shuttle vector, an expression vector, an extrachromosomal vector, and so forth. Means for delivering or introducing various vectors into Bacteroides are well known in the art.

The nucleic acid sequence encoding a CRISPR-base editor fusion can be operably linked to a promoter for expression in the bacteria of interest. In specific embodiments, sequence encoding a CRISPR-base editor fusion can be operably linked to a regulated promoter. In some aspects, the regulated promoter can be regulated by a promoter inducing chemical. In such embodiments, the promoter can be pTetO, which is based on the Escherichia coli Tn10-derived tet regulatory system and consists of a strong tet operator (tetO)-containing mycobacterial promoter and expression cassette for the repressor TetR) and the promoter inducing chemical can be anhydrotetracycline (aTc). In other embodiments, the promoter can be pBAD or araC-ParaBAD and the promoter inducing chemical can be arabinose. In further embodiments, the promoter can be pLac or tac (trp-lac) and the promoter inducing chemical can be lactose/IPTG. In other embodiments, the promoter can be pPrpB and the promoter inducing chemical can be propionate.

The nucleic acid sequence encoding the at least one guide RNA can be operably linked to a promoter for expression in the bacteria of interest. In general, expression of the at least one guide RNA can be regulated by constitutive promoters. In embodiments in which the bacteria of interest is Bacteroides, the constitutive promoter can be the P1 promoter, which lies upstream of the B. thetaiotaomicron 16S rRNA gene BT_r09 (Wegmann et al., Applied Environ. Microbiol., 2013, 79:1980-1989). Other suitable Bacteroides promoters include P2, P1TD, P1Tp, P1TDP (Lim et al., Cell, 2017, 169:547-558), P_{AM}, P_{cfiA}, P_{cepA}, P_{BT1311} (Mimee et al., Cell Systems, 2015, 1:62-71) or variants of any of the foregoing promoters. In other embodiments, the constitutive promoter can be an E. coli σ⁷⁰ promoter or derivative thereof, a B. subtilis σ^{A} promoter or derivative thereof, or a Salmonella Pspv2 promoter or derivative thereof. Persons skilled in the art are familiar with additional constitutive promoters that are suitable for the bacteria of interest.

In some embodiments, the vector can be an integrative vector and can further comprise sequence encoding a recombinase, as well as one or more recombinase recognition sites. In general, the recombinase is an irreversible recombinase. Non-limiting examples of suitable recombinases include the Bacteroides intN2 tyrosine integrase (coded by NBU2 gene), Streptomyces phage phiC31 (ϕC31) recombinase, coliphage P4 recombinase, coliphage lambda integrase, Listeria A118 phage recombinase, and actinophage R4 Sre recombinase. Recombinases/integrases mediate recombination between two sequence specific recognition (or attachment) sites (e.g., an attP site and an attB site). In some embodiments, the vector can comprise one of the recombinase recognition sites (e.g., attP) and the other recombinase recognition site (e.g., attB) can be located in the chromosome of the bacteria (e.g., near a tRNA-Ser gene). In such situations, the entire vector can be integrated into the chromosome of the bacteria. In other embodiments, the sequence encoding the engineered CRISPR nucleobase modifying system can be flanked by the two recombinase recognition sites, such that only the sequence encoding the engineered CRISPR nucleobase modifying system is integrated into the bacterial chromosome.

Any of the vectors described above can further comprise at least one transcriptional termination sequence, as well as at least one origin of replication and/or at least one selectable marker sequence (e.g., antibiotic resistance genes) for propagation and selection in Bacteroides cells of interest.

Additional information about vectors and use thereof can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

In embodiments in which the vector encoding the engineered CRISPR nucleobase modifying system is an integrative vector, the nucleic acid encoding the engineered system (or the entire vector) can be stably integrated into the Bacteroides chromosome after delivery of the vector to the organism (and expression of the recombinase/integrase). In embodiments in which the vector encoding the engineered CRISPR nucleobase modifying system is not an integrative vector, the vector can remain extrachromosomal after delivery of the vector to the bacteria.

In embodiments in which the nucleic acid sequence encoding a CRISPR-base editor fusion is operably linked to an inducible promoter, expression of the CRISPR nucleobase modifying system can be induced by introducing a promoter inducing chemical into the bacteria. In specific embodiments, the promoter inducing chemical can be anhydrotetracycline. Upon induction, the CRISPR-base editor fusion is synthesized and complexes with the at least one guide RNA, which targets the CRISPR nucleobase modifying system to the target locus in the bacterial chromosome, thereby forming the protein-nucleic acid complex as disclosed herein.

### (III) Methods for Modifying Nucleobases in Bacteria

A further aspect of the present disclosure encompasses methods for modifying at least one nucleobase in a chromosome of a target member of Bacteroidetes. The method comprises expressing an engineered RNA-guided (CRISPR) nucleobase modifying system in the target species/strain, wherein the engineered RNA-guided (CRISPR) nucleobase modifying system is targeted to a specific locus in a chromosome of the target bacteria and the engineered RNA-guided nucleobase modifying system modifies at least one nucleobase within the specific locus, such that a gene comprising the specific locus is modified and/or inactivated, and wherein the chromosome of the target bacterial species/strain encodes an HU family DNA-binding protein comprising an amino acid sequence with at least 50% sequence identity to SEQ ID NO: 1 (e.g., at least 55%, at least 60%, at least 65%, at least 70%, at least 75%, at least 80%, at least 85%, at least 90%, at least 95%, or at least 99% sequence identity to SEQ ID NO: 1). The nucleobase modifications (e.g., conversion of cytosine to thymine or adenine to guanine) can introduce single nucleotide polymorphisms (SNPs) and/or stop codons within the specific locus. As a consequence of the at least one nucleobase modification, the target bacteria can have altered, reduced, or eliminated expression of at least one gene comprising the specific locus.

Any of the RNA-guided (CRISPR) nucleobase modification systems described above in section (I)(a) can be engineered as described above in section (II) to target a specific locus in the chromosome of a bacterial species/strain in a Bacteroidetes phylogenetic lineage of interest, which are described above in section (I)(b). The engineered CRISPR nucleobase modification system can be introduced into the bacteria as part of a vector as described above in section (II). In general, the CRISPR-nucleobase modification system is inducible (e.g., nucleic acid sequence encoding a CRISPR-base editor fusion is operably linked to an inducible promoter). As such, the CRISPR nucleobase modification system can be expressed at a defined point in time. In the absence of a promoter inducing chemical, the CRISPR nucleobase modification system cannot be generated. A CRISPR-base editor fusion can be produced by exposing the bacteria to a promoter inducing chemical, such that the CRISPR-base editor fusion protein is expressed from the chromosomally integrated encoding sequence or the extrachromosomal encoding sequence as described above in section (II). The CRISPR-base editor fusion complexes with the at least one guide RNA that is constitutively expressed from the chromosomally integrated encoding sequence or the extrachromosomal encoding sequence, thereby forming an active CRISPR nucleobase modification system. The CRISPR nucleobase modification system is targeted to the specific locus in the bacterial chromosome, where it modifies at least one nucleobase, such that expression of a gene comprising the specific locus is altered, reduced, or eliminated.

In some embodiments, the target organism can be a Bacteroides species or strain level variant, as detailed above in section (I)(b).

In other embodiments, the organism can be harbored in a mammal's digestive tract (or gut), wherein administration of the promoter inducing chemical can lead to nucleobase modifications (e.g., conversion of cytosine to thymine or adenine to guanine) that may lead to reduced or eliminated levels of the target bacteria in the gut microbiota. The promoter inducing chemical can be administered orally (e.g., via food, drink, or a pharmaceutical formulation). The mammal can be a mouse, rat, or other research animal. In specific embodiments, the mammal can be a human. Reduction or elimination of the target bacterial organism (e.g., a member of the genus Bacteroides), for example, can lead to improved gut health.

The mixed population of bacteria (in cell culture or a digestive tract) can comprise a wide diversity of taxa. For example, human gut microbiota can comprise hundreds of different species of bacteria with accompanying substantial strain level diversity.

In certain embodiments, the mammal (e.g., human) can be undergoing cancer immunotherapy, wherein immunotherapy responders have been shown to have lower levels of Bacteroides species in their gut microbiota as compared to non-responders (Gopalakrishnan et al., Science, 2018, 359:97-103). Thus, reduction in the levels of Bacteroides species in gut microbiota may lead to better human cancer immunotherapy outcomes.

In certain embodiments, the mammal (e.g., human, canine, feline, porcine, equine, or bovine) can undergo gut surgery for a variety of reasons including, but not limited to, inflammatory bowel disease, Crohn's disease, diverticulitis, bowel blockage, polyp removal, cancerous tissue removal, ulcerative colitis, bowel resection, proctectomy, complete colectomy, or partial colectomy wherein attenuation of Bacteroides fragilis species within the mammalian gut pre-surgery by an inducible CRISPR nucleobase modification system may reduce the risk of post-surgery infections by B. fragilis at locations outside the gut, but within the mammalian body. Locations outside the gut include the external surface of the gut. The inducible CRISPR nucleobase modification systems within B. fragilis can be targeted to modify a location similar, but not limited to, a pathogenicity island, toxins (i.e., B. fragilis toxin or BFT) or other unique sequence associated with infectious strains of B. fragilis or other native gut bacteria known to cause post-surgical infections. For example, levels of nontoxigenic B. fragilis (NTBF) and enterotoxigenic B. fragilis (ETBF) may be selectively modulated using engineered inducible CRISPR nucleobase modification systems placed within ETBF strains, but not NTBF strains. Other gut bacteria at risk for causing infections after gut surgery may include Bacteroides capillosis, Escherichia coli, Enterococcus faecalis, Gamella haemolysan, and Morganella morganii. Delivery of the inducible CRISPR nucleobase modification system to the gut microbiota may occur as part of a probiotic treatment before, during, or after surgery. Delivery of the inducible CRISPR nucleobase modification system to the target bacteria may occur outside the mammalian body or within the mammalian body. Delivery of the inducible CRISPR nucleobase modification system to the target bacteria may occur via nucleic acid vectors such as plasmids or bacteriophage. Delivery of plasmids may occur via electroporation, chemical transformation, or bacteria-to-bacteria conjugation.

### (IV) CRISPR Integrated Bacterial Species/Strains as Probiotics

Yet another aspect of the present disclosure encompasses engineered bacterial strains for use, e.g., as probiotics. The engineered strains comprise any of engineered CRISPR nucleobase modification systems described in section (I)(a) integrated into the bacterial chromosome or maintained as episomal vectors within the organism of interest. In some embodiments, the engineered bacteria is an engineered Bacteroides comprising an inducible CRISPR nucleobase modification system. Administration of the engineered Bacteroides to a mammalian subject followed by induction of the CRISPR system can be used to target a specific locus in the bacterial chromosome. Modification of at least one nucleobase by this CRISPR system, such that expression of a gene comprising the specific locus is altered, reduced or eliminated, thereby, provides a therapeutic benefit to the mammalian subject. In other embodiments, Bacteroides strains can be engineered to out-compete wildtype strains of Bacteroides in gut microbiota. In these and other embodiments, engineered Bacteroides strains providing a therapeutic benefit for the mammalian subject can then be removed from the mammalian subject by induction of the inducible CRISPR nucleobase modification system.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “about” when used in relation to a numerical value, x, for example means x±5%.

As used herein, the terms “complementary” or “complementarity” refer to the association of double-stranded nucleic acids by base pairing through specific hydrogen bonds. The base paring may be standard Watson-Crick base pairing (e.g., 5′-A G T C-3′ pairs with the complementary sequence 3′-T C A G-5′). The base pairing also may be Hoogsteen or reversed Hoogsteen hydrogen bonding. Complementarity is typically measured with respect to a duplex region and thus, excludes overhangs, for example. Complementarity between two strands of the duplex region may be partial and expressed as a percentage (e.g., 70%), if only some (e.g., 70%) of the bases are complementary. The bases that are not complementary are “mismatched.” Complementarity may also be complete (i.e., 100%), if all the bases in the duplex region are complementary.

The term “expression” with respect to a gene or polynucleotide refers to transcription of the gene or polynucleotide and, as appropriate, translation of an mRNA transcript to a protein or polypeptide. Thus, as will be clear from the context, expression of a protein or polypeptide results from transcription and/or translation of the open reading frame.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “heterologous” refers to an entity that is not endogenous or native to the cell of interest. For example, a heterologous protein refers to a protein that is derived from or was originally derived from an exogenous source, such as an exogenously introduced nucleic acid sequence. In some instances, the heterologous protein is not normally produced by the cell of interest.

The term “nickase” refers to an enzyme that cleaves one strand of a double-stranded nucleic acid sequence.

The term “nuclease,” which is used interchangeably with the term “endonuclease,” refers to an enzyme that cleaves both strands of a double-stranded nucleic acid sequence or cleaves a single-stranded nucleic acid sequence.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine), nucleotide isomers, or nucleotide analogs. A nucleotide analog refers to a nucleotide having a modified purine or pyrimidine base or a modified ribose moiety. A nucleotide analog may be a naturally occurring nucleotide (e.g., inosine, pseudouridine, etc.) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

The terms “target sequence,” “target site” and “specific locus) are used interchangeably to refer to the specific sequence in the nucleic acid of interest (e.g., chromosomal DNA or cellular RNA) to which the CRISPR system is targeted, and the site at which the CRISPR system modifies the nucleic acid or protein(s) associated with the nucleic acid.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website.

As various changes could be made in the above-described cells and methods without departing from the scope of the invention, it is intended that all matter contained in the above description and in the examples given below, shall be interpreted as illustrative and not in a limiting sense.

### EXAMPLES

The following examples illustrate certain aspects of the disclosure.

### Example 1. CRISPR Base Editing in Bacteroides thetaiotaomicron

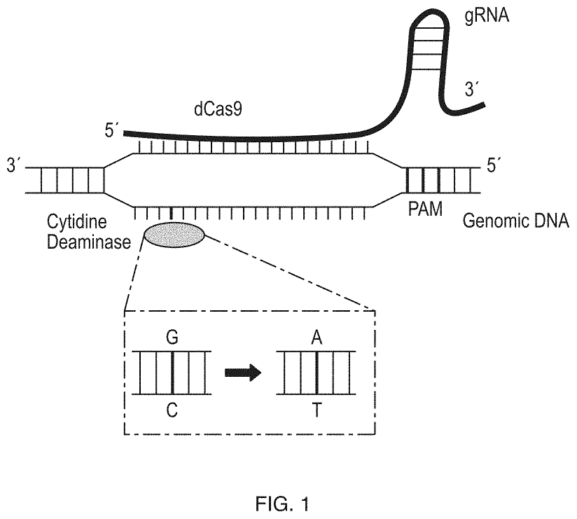

Deaminase-mediated targeted base editing in Bacteroides was conducted to directly edit nucleotides at the target locus, specified by a guide RNA, without DNA cleavage or a template donor DNA (FIG. 1). Nearly 100% editing efficiency was achieved without inducing cell death and thus is suitable for genome engineering of Bacteroides.

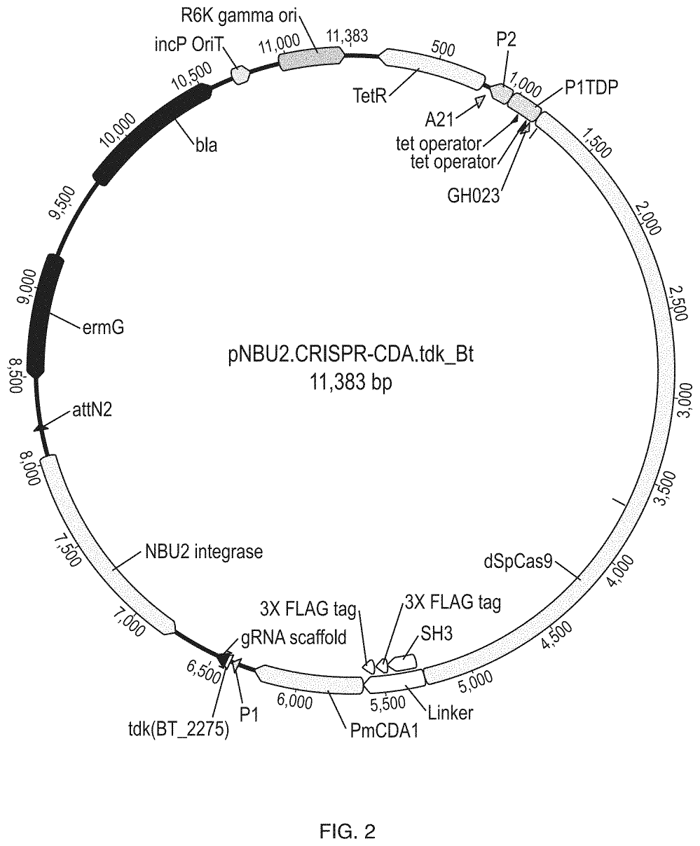

A Bacteroides dCas9-AID vector pNBU2.CRISPR-CDA was constructed. The vector expresses (i) a catalytically inactivated Cas9 (dCas: D10A and H840A mutations) fused to Petromyzon marinus cytosine deaminase PmCDA1 (CDA) under an anhydrotetracycline-inducible promoter and (ii) a 20-nucleotide (nt) target sequence-gRNA scaffold hybrid (sgRNA) under a constitutive promoter PI. The plasmid contains an R6K origin of replication and bla sequence for ampicillin selection in E. coli, RP4-oriT sequence for conjugation and ermG sequence for erythromycin (Em) selection in Bacteroides. NBU2 encodes the intN2 tyrosine integrase which mediates sequence-specific recombination between the attN2 site on pNBU2.CRISPR-CDA plasmid and one of the attB sites located on the chromosome of Bacteroides cells (Wang et al., J. Bacteriology, 2000, 182(12):3559-3571). The NBU2 integrase recognition sequence (attN2/attB) is 5′-CCTGTCTCTCCGC-3′ (SEQ ID NO: 2). The CRISPR-CDA unit consists of inducible, nuclease-deficient SpCas9 with D10A and H840A mutations fused with Petromyzon marinus cytosine deaminase (PmCDA1). The dCas9-CDA1 fusion was controlled by TetR regulator (P2-A21-tetR, P1TDP-GH023-dSpCas9-PmCDA1) under the control of anhydrotetracycline (aTc), and the guide RNA was controlled by constitutive P1 promoter (P1-N20 sgRNA scaffold). The promoters and ribosomal binding sites are derived and engineered from regulatory sequences of Bacteroides thetaiotaomicron (Bt) 16S rRNA genes, as described in Lim et al., Cell, 2017, 169:547-558. The guide RNA is a nucleotide sequence that is homologous to a coding or non-coding DNA sequence or is a non-targeting scramble nucleotide sequence. This sequence can vary as long as it is compatible with protospacer adjacent motif (PAM) requirements of different Cas9 homologs. The guide RNA can be either in separate transcriptional units of tracrRNA and crRNA or fused into a hybrid chimeric tracr/crRNA single guide (sgRNA). A map of plasmid pNBU2.CRISPR-STOP.tdkfit DNA sequence (11, 383 bp) is shown in FIG. 2. and listed as SEQ ID NO: 3:

GGAAAGCGGGCAGTGAGCGCAACGCAATTAATGTGAGTTAGCTCACTCA
TTAGGCACCCCAGGCTTTACACTTTATGCTTCCGGCTCGTATGTTGTGTG
GAATTGTGAGCGGATAACAATTTCACACAGGAAACAGCTATGACCATGAT
TACGCCCTTAAGACCCACTTTCACATTTAAGTTGTTTTTCTAATCCGCATA
TGATCAATTCAAGGCCGAATAAGAAGGCTGGCTCTGCACCTTGGTGATC
AAATAATTCGATAGCTTGTCGTAATAATGGCGGCATACTATCAGTAGTAG
GTGTTTCCCTTTCTTCTTTAGCGACTTGATGCTCTTGATCTTCCAATACGC
AACCTAAAGTAAAATGCCCCACAGCGCTGAGTGCATATAATGCATTCTCT
AGTGAAAAACCTTGTTGGCATAAAAAGGCTAATTGATTTTCGAGAGTTTC
ATACTGTTTTTCTGTAGGCCGTGTACCTAAATGTACTTTTGCTCCATCGC
GATGACTTAGTAAAGCACATCTAAAACTTTTAGCGTTATTACGTAAAAAAT
CTTGCCAGCTTTCCCCTTCTAAAGGGCAAAAGTGAGTATGGTGCCTATCT
AACATCTCAATGGCTAAGGCGTCGAGCAAAGCCCGCTTATTTTTTACATG
CCAATACAATGTAGGCTGCTCTACACCTAGCTTCTGGGCGAGTTTACGG
GTTGTTAAACCTTCGATTCCGACCTCATTAAGCAGCTCTAATGCGCTGTT
AATCACTTTACTTTTATCTAATCTAGACATATTCGTTTAATATCATAAATA
ATTTATTTTATTTTAAAATGCGCGGGTGCAAAGGTAAGAGGTTTTATTTTA
ACTACCAAATGTTTTCGGAAGTTTTTTCGCTTTTCTTTTTCTATCGTTTCT
CAGACTCTCTTAGCGAAAGGGAAAGAAGGTAAAGAAGAAAAACAAAACGCC
TTTTCTTTTTTGCACCCGCTTTCCAAGAGAAGAAAGCCTTGTTAAATTGAC
TTAGTGTAAAAGCGCAGTACTGCTTGACCATAAGAACAAAAAAATCTCTA
TCACTGATAGGGATAAAGTTTGGAAGATAAAGCTAAAAGTTCTTATCTTTG
CAGTCTCCCTATCAGTGATAGAGACGAAATAAAGACATATAAAAGAAAAG
ACACCATGGATAAGAAATACTCAATAGGCTTAGCTATCGGCACAAATAGC
GTCGGATGGGCGGTGATCACTGATGAATATAAGGTTCCGTCTAAAAAGT
TCAAGGTTCTGGGAAATACAGACCGCCACAGTATCAAAAAAAATCTTATA
GGGGCTCTTTTATTTGACAGTGGAGAGACAGCGGAAGCGACTCGTCTCA
AACGGACAGCTCGTAGAAGGTATACACGTCGGAAGAATCGTATTTGTTAT
CTACAGGAGATTTTTTCAAATGAGATGGCGAAAGTAGATGATAGTTTCTT
TCATCGACTTGAAGAGTCTTTTTTGGTGGAAGAAGACAAGAAGCATGAAC
GTCATCCTATTTTTGGAAATATAGTAGATGAAGTTGCTTATCATGAGAAAT
ATCCAACTATCTATCATCTGCGAAAAAAATTGGTAGATTCTACTGATAAAG
CGGATTTGCGCTTAATCTATTTGGCCTTAGCGCATATGATTAAGTTTCGT
GGTCATTTTTTGATTGAGGGAGATTTAAATCCTGATAATAGTGATGTGGA
CAAACTATTTATCCAGTTGGTACAAACCTACAATCAATTATTTGAAGAAAA
CCCTATTAACGCAAGTGGAGTAGATGCTAAAGCGATTCTTTCTGCACGAT
TGAGTAAATCAAGACGATTAGAAAATCTCATTGCTCAGCTCCCCGGTGAG
AAGAAAAATGGCTTATTTGGGAATCTCATTGCTTTGTCATTGGGTTTGAC
CCCTAATTTTAAATCAAATTTTGATTTGGCAGAAGATGCTAAATTACAGCT
TTCAAAAGATACTTACGATGATGATTTAGATAATTTATTGGCGCAAATTGG
AGATCAATATGCTGATTTGTTTTTGGCAGCTAAGAATTTATCAGATGCTAT
TTTACTTTCAGATATCCTAAGAGTAAATACTGAAATAACTAAGGCTCCCCT
ATCAGCTTCAATGATTAAACGCTACGATGAACATCATCAAGACTTGACTC
TTTTAAAAGCTTTAGTTCGACAACAACTTCCAGAAAAGTATAAAGAAATCT
TTTTTGATCAATCAAAAAACGGATATGCAGGTTATATTGATGGGGGAGCT
AGCCAAGAAGAATTTTATAAATTTATCAAACCAATTTTAGAAAAAATGGAT
GGTACTGAGGAATTATTGGTGAAACTAAATCGTGAAGATTTGCTGCGCAA
GCAACGGACCTTTGACAACGGCTCTATTCCCCATCAAATTCACTTGGGTG
AGCTGCATGCTATTTTGAGAAGACAAGAAGACTTTTATCCATTTTTAAAAG
ACAATCGTGAGAAGATTGAAAAAATCTTGACTTTTCGAATTCCTTATTATG
TTGGTCCATTGGCGCGTGGCAATAGTCGTTTTGCATGGATGACTCGGAA
GTCTGAAGAAACAATTACCCCATGGAATTTTGAAGAAGTTGTCGATAAAG
GTGCTTCAGCTCAATCATTTATTGAACGCATGACAAACTTTGATAAAAATC
TTCCAAATGAAAAAGTACTACCAAAACATAGTTTGCTTTATGAGTATTTTA
CGGTTTATAACGAATTGACAAAGGTCAAATATGTTACTGAAGGAATGCGA
AAACCAGCATTTCTTTCAGGTGAACAGAAGAAAGCCATTGTTGATTTACT
CTTCAAAACAAATCGAAAAGTAACCGTTAAGCAATTAAAAGAAGATTATTT
CAAAAAAATAGAATGTTTTGATAGTGTTGAAATTTCAGGAGTTGAAGATAG
ATTTAATGCTTCATTAGGTACCTACCATGATTTGCTAAAAATTATTAAAGA
TAAAGATTTTTTGGATAATGAAGAAAATGAAGATATCTTAGAGGATATTGT
TTTAACATTGACCTTATTTGAAGATAGGGAGATGATTGAGGAAAGACTTA
AAACATATGCTCACCTCTTTGATGATAAGGTGATGAAACAGCTTAAACGT
CGCCGTTATACTGGTTGGGGACGTTTGTCTCGAAAATTGATTAATGGTAT
TAGGGATAAGCAATCTGGCAAAACAATATTAGATTTTTTGAAATCAGATG
GTTTTGCCAATCGCAATTTTATGCAGCTGATCCATGATGATAGTTTGACAT
TTAAAGAAGACATTCAAAAAGCACAAGTGTCTGGACAAGGCGATAGTTTA
CATGAACATATTGCAAATTTAGCTGGTAGCCCTGCTATTAAAAAAGGTATT
TTACAGACTGTAAAAGTTGTTGATGAATTGGTCAAAGTAATGGGGCGGCA
TAAGCCAGAAAATATCGTTATTGAAATGGCACGTGAAAATCAGACAACTC
AAAAGGGCCAGAAAAATTCGCGAGAGCGTATGAAACGAATCGAAGAAGG
TATCAAAGAATTAGGAAGTCAGATTCTTAAAGAGCATCCTGTTGAAAATA
CTCAATTGCAAAATGAAAAGCTCTATCTCTATTATCTCCAAAATGGAAGAG
ACATGTATGTGGACCAAGAATTAGATATTAATCGTTTAAGTGATTATGATG
TCGATGCCATTGTTCCACAAAGTTTCCTTAAAGACGATTCAATAGACAATA
AGGTCTTAACGCGTTCTGATAAAAATCGTGGTAAATCGGATAACGTTCCA
AGTGAAGAAGTAGTCAAAAAGATGAAAAACTATTGGAGACAACTTCTAAA
CGCCAAGTTAATCACTCAACGTAAGTTTGATAATTTAACGAAAGCTGAAC
GTGGAGGTTTGAGTGAACTTGATAAAGCTGGTTTTATCAAACGCCAATTG
GTTGAAACTCGCCAAATCACTAAGCATGTGGCACAAATTTTGGATAGTCG
CATGAATACTAAATACGATGAAAATGATAAACTTATTCGAGAGGTTAAAGT
GATTACCTTAAAATCTAAATTAGTTTCTGACTTCCGAAAAGATTTCCAATT
CTATAAAGTACGTGAGATTAACAATTACCATCATGCCCATGATGCGTATC
TAAATGCCGTCGTTGGAACTGCTTTGATTAAGAAATATCCAAAACTTGAAT
CGGAGTTTGTCTATGGTGATTATAAAGTTTATGATGTTCGTAAAATGATTG
CTAAGTCTGAGCAAGAAATAGGCAAAGCAACCGCAAAATATTTCTTTTAC
TCTAATATCATGAACTTCTTCAAAACAGAAATTACACTTGCAAATGGAGAG
ATTCGCAAACGCCCTCTAATCGAAACTAATGGGGAAACTGGAGAAATTGT
CTGGGATAAAGGGCGAGATTTTGCCACAGTGCGCAAAGTATTGTCCATG
CCCCAAGTCAATATTGTCAAGAAAACAGAAGTACAGACAGGCGGATTCT
CCAAGGAGTCAATTTTACCAAAAAGAAATTCGGACAAGCTTATTGCTCGT
AAAAAAGACTGGGATCCAAAAAAATATGGTGGTTTTGATAGTCCAACGGT
AGCTTATTCAGTCCTAGTGGTTGCTAAGGTGGAAAAAGGGAAATCGAAG
AAGTTAAAATCCGTTAAAGAGTTACTAGGGATCACAATTATGGAAAGAAG
TTCCTTTGAAAAAAATCCGATTGACTTTTTAGAAGCTAAAGGATATAAGGA
AGTTAAAAAAGACTTAATCATTAAACTACCTAAATATAGTCTTTTTGAGTT
AGAAAACGGTCGTAAACGGATGCTGGCTAGTGCCGGAGAATTACAAAAAG
GAAATGAGCTGGCTCTGCCAAGCAAATATGTGAATTTTTTATATTTAGCTA
GTCATTATGAAAAGTTGAAGGGTAGTCCAGAAGATAACGAACAAAAACAA
TTGTTTGTGGAGCAGCATAAGCATTATTTAGATGAGATTATTGAGCAAAT
CAGTGAATTTTCTAAGCGTGTTATTTTAGCAGATGCCAATTTAGATAAAGT
TCTTAGTGCATATAACAAACATAGAGACAAACCAATACGTGAACAAGCAG
AAAATATTATTCATTTATTTACGTTGACGAATCTTGGAGCTCCCGCTGCTT
TTAAATATTTTGATACAACAATTGATCGTAAACGATATACGTCTACAAAAG
AAGTTTTAGATGCCACTCTTATCCATCAATCCATCACTGGTCTTTATGAAA
CACGCATTGATTTGAGTCAGCTAGGAGGTGACGGTGGAGGAGGTTCTG
GAGGTGGAGGTTCTGCTGAGTATGTGCGAGCCCTCTTTGACTTTAATGG
GAATGATGAAGAGGATCTTCCCTTTAAGAAAGGAGACATCCTGAGAATCC
GGGATAAGCCTGAGGAGCAGTGGTGGAATGCAGAGGACAGCGAAGGAA
AGAGGGGGATGATTCCTGTCCCTTACGTGGAGAAGTATTCCGGAGACTA
TAAGGACCACGACGGAGACTACAAGGATCATGATATTGATTACAAAGAC
GATGACGATAAGTCTAGGCTCGAGTCCGGAGACTATAAGGACCACGACG
GAGACTACAAGGATCATGATATTGATTACAAAGACGATGACGATAAGTCT
AGGATGACCGACGCTGAGTACGTGAGAATCCATGAGAAGTTGGACATCT
ACACGTTTAAGAAACAGTTTTTCAACAACAAAAAATCCGTGTCGCATAGA
TGCTACGTTCTCTTTGAATTAAAACGACGGGGTGAACGTAGAGCGTGTTT
TTGGGGCTATGCTGTGAATAAACCACAGAGCGGGACAGAACGTGGCATT
CACGCCGAAATCTTTAGCATTAGAAAAGTCGAAGAATACCTGCGCGACA
ACCCCGGACAATTCACGATAAATTGGTACTCATCCTGGAGTCCTTGTGCA
GATTGCGCTGAAAAGATCTTAGAATGGTATAACCAGGAGCTGCGGGGGA
ACGGCCACACTTTGAAAATCTGGGCTTGCAAACTCTATTACGAGAAAAAT
GCGAGGAATCAAATTGGGCTGTGGAATCTCAGAGATAACGGGGTTGGGT
TGAATGTAATGGTAAGTGAACACTACCAATGTTGCAGGAAAATATTCATC
CAATCGTCGCACAATCAATTGAATGAGAATAGATGGCTTGAGAAGACTTT
GAAGCGAGCTGAAAAACGACGGAGCGAGTTGTCCATTATGATTCAGGTA
AAAATACTCCACACCACTAAGAGTCCTGCTGTTTAAATTAATGCGGCTGC
AATTTTTTTGGGCGGGGCCGCCCAAAAAAATCCTAGCACCCTGCAGCAG
TACTGCTTGACCATAAGAACAAAAAAACTTCCGATAAAGTTTGGAAGATA
AAGCTAAAAGTTCTTATCTTTGCAGTATACAAGAGACCAGAAGAAGGTTT
TAGAGCTAGAAATAGCAAGTTAAAATAAGGCTAGTCCGTTATCAACTTGA
AAAAGTGGCACCGAGTCGGTGCTTTTTTTGAGATCTGTCGACTCTAGAG
GATCCCCGGGTACCGAGCTCGAATTCACTGGCCGTCGTTTTACAACGTC
GTGACTGGGAAAACCCTGGCGTTACCCAACTTAATCGTACTTGTGCCTG
TTCTATTTCCGAACCGACCGCTTGTATGAATCCATCAAAATTCGTTTTCTC
TATGTTGGATTCCTTGTTGCTCATATTGTGATGATAATTTCTACAAATATA
GTCATTGGTAACTATCTATGAAACTGTTTGATACTTTTATAGTTGATTAAA
CTTGTTCATGGCATTTGCCTTAATATCATCCGCTATGTCAATGTAGGGTTT
CATAGCTTTGTAGTCGCTGTGTCCCGTCCATTTCATGACCACCTGTGCCG
GGATTCCGAGAGCCAGCGCATTGCAGATGAATGTCCTTCTTCCTGCATG
GGTACTGAGCAAAGCGTATTTGGGTGTGACTTCATCAATACGTTCATTTC
CCTTGTAGTAGGTTTCCCGTACAGGCTCGTTGATTTCTGCCAGTTCGCCC
AGCTCTTTCAGGTAATCGTTCATCTTCTGGTTGCTGATGACGGGCAGAG
CCATGTAATTCTCGAAATGGATGTCCTTGTATTTGTCCAGTATGGCTTTG
CTGTATTTGTTCAGTTCAATCGTCAGGCTGTCGGCAGTCTTGACTGTGGT
TATTTCGATGTGGTCGGACTTCACATCGCTTCTTTTCAGATTGCGAACAT
CCGAATACCGCAAACTCGTAAAGCAGCAGAACAGGAAAACATCACGCAC
ACGTTCCAGGTATTGCTTATCCTTGGGTATCTGGTAGTCTTTCAGCTTGT
TCAGTTCATCCCAAGTCAGGAAGATTACTTTTTTCGAGGTGGTTTTCAGT
TTCGGTTTGAACGTATCGTATGCAATGTTCTGATGATGTCCTTTCTTGAA
GCTCCAGCGCAGGAACCATTTGAGGAATCCCATTTGCTTGCCGATGGTG
CTGTTTCTCATATCCTTGGTGTCACGCAGGAAGTTGACGTATTCGTTCAA
TCCAAACTCGTTGAAATAGTTGAACGTTGCATCCTCCTTGAACTCTTTGA
GGTGGTTCCTCACTGCTGCAAATTTTTCATAGGTGGATGCCGTCCAGTTA
TTCTGGTTACCGCACTCTTTTACAAACTCATCGAACACCTCCCAAAAGCT
GACAGGGGCTTCTTCCGGCTGTTCTTCGCTGGTGTCTTTCATTCTCATGT
TGAAAGCTTCCTTCAACTGTTGGGTCGTTGGCATGACCTCCTGCACCTCA
AATTCCTTGAAAATATTCTGGATTTCGGCATAGTATTTCAGCAAGTCCGTA
TTGATTTCGGCTGCACTTTGCTTTAGCTTGTTGGTACATCCGCTCTTTACC
CGCTGCTTATCTGCATCCCATTTGGCTACGTCAATCCGGTAGCCCGTTGT
AAACTCGATGCGTTGGCTGGCAAAGATGACACGCATACGGATGGGTACG
TTCTCTACGATTGGCACACCGTTCTTTTTCCGGCTCTCCAATGCAAAAAT
GATGTTGCGCTTGATATTCATAATTGGGTGCGTTTGAAATTCTACACCCA
AATATACACCCAATTATTGAGATAGCAAAAGACATTTAGAAACATTTACTT
TTACTCTATATTGTAATTTACACTTGATTATCAGTCGTTTGCAGTCTTATG
ATATTCTGTGAAAGTATAAGTTCGAGAGCCTGTCTCTCCGCAAAAAACGCT
GAAAATCAGCAGATTGCAAAACAAACACCCTGTTTTACACCCAAGAATGT
AAAGTCGGCTGTTTTTGTTTTATTTAAGATAATACAACCACTACATAATAA
AAGAGTAGCGATATTAAAAGAATCCGATGAGAAAAGACTAATATTTATCTA
TCCATTCAGTTTGATTTTTCAGGACTTTACATCGTCCTGAAAGTATTTGTT
GGTACCGGTACCGAGGACGCGTAAACATTTACAGTTGCATGTGGCCTAT
TGTTTTTAGCCGTTAAATATTTTATAACTATTAAATAGCGATACAAATTGT
TCGAAACTAATATTGTTTATATCATATATTCTCGCATGTTTTAAAGCTTTA
TTAAATTGATTTTTTGTAAACAGTTTTTCGTACTCTTTGTTAACCCATTTC
ATTACAAAAGTTTCATATTTTTTTCTCTCTTTAAATGCCATTTTTGCTGGC
TTTCTTTTTAATACAATTAATGTGCTATCCACTTTAGGTTTTGGATGGAAA
TAATACCTAGGAATTTTTGCTAATATAGAAATATCTACCTCTGCCATTAAC
AGCAATGCTAGTGATCTGTTTGTATCTAATAACATTTTAGCAAAACCATAT
TCCACTATTAAATAACTTATTGTGGCTGAACTTTCAAAAACAATTTTTCGA
ATTATATTTGTGCTTATGTTGTAAGGTATGCTGCCAAATATTTTATATGGA
TTGTGGCTAGGAAATGTAAATTTCAGTATATCATCATTTACTATTTGATAG
TTAGGATAATTTAAGAGCTTATTACGAGTTACCTCACATAATTTAGAATCA
ATTTCTATCGCCGTTACAAAATTACATCTCTTTACCAATCCAGCAGTAAAA
TGACCTTTCCCTGCACCTATTTCAAAGATGTTATCTTTTTCATCTAAACTT
ATGCAATTCATTATTTTTTCTATGTGATATTTTGAAGTAATAAAATTTTGA
CTATCTTTTATATTTACTTTGTTCATTATAACCTCTCCTTAATTTATTGCA
TCTCTTTTCGAATATTTATGTTTTTTGAGAAAAGAACGTACTCATGGTTCA
TCCCGATATGCGTATCGGTCTGTATATCAGCAACTTTCTATGTGTTTCAAC
TACAATAGTCATCTATTCTCATCTTTCTGAGTCCACCCCCTGCAAAGCCCC
TCTTTACGACATAAAAATTCGGTCGGAAAAGGTATGCAAAAGATGTTTCTC
TCTTTAAGAGAAACTCTTCGGGATGCAAAAATATGAAAATAACTCCAATTC
ACCAAATTATATAGCGACTTTTTTACAAAATGCTAAAATTTGTTGATTTCC
GTCAAGCAATTGTTGAGCAAAAATGTCTTTTACGATAAAATGATACCTCAA
TATCAACTGTTTAGCAAAACGATATTTCTCTTAAAGAGAGAAACACCTTTT
TGTTCACCAATCCCCGACTTTTAATCCCGCGGCCATGATTGAAAAAGGAAG
AGTATGAGTATTCAACATTTCCGTGTCGCCCTTATTCCCTTTTTTGCGGCA
TTTTGCCTTCCTGTTTTTGCTCACCCAGAAACGCTGGTGAAAGTAAAAGAT
GCTGAAGATCAGTTGGGTGCACGAGTGGGTTACATCGAACTGGATCTCAAC
AGCGGTAAGATCCTTGAGAGTTTTCGCCCCGAAGAACGTTTTCCAATGATG
AGCACTTTTAAAGTTCTGCTATGTGGCGCGGTATTATCCCGTATTGACGCC
GGGCAAGAGCAACTCGGTCGCCGCATACACTATTCTCAGAATGACTTGGTT
GAGTACTCACCAGTCACAGAAAAGCATCTTACGGATGGCATGACAGTAA
GAGAATTATGCAGTGCTGCCATAACCATGAGTGATAACACTGCGGCCAA
CTTACTTCTGACAACGATCGGAGGACCGAAGGAGCTAACCGCTTTTTTG
CACAACATGGGGGATCATGTAACTCGCCTTGATCGTTGGGAACCGGAGC
TGAATGAAGCCATACCAAACGACGAGCGTGACACCACGATGCCTGTAGC
AATGGCAACAACGTTGCGCAAACTATTAACTGGCGAACTACTTACTCTAG
CTTCCCGGCAACAATTAATAGACTGGATGGAGGCGGATAAAGTTGCAGG
ACCACTTCTGCGCTCGGCCCTTCCGGCTGGCTGGTTTATTGCTGATAAA
TCTGGAGCCGGTGAGCGTGGGTCTCGCGGTATCATTGCAGCACTGGGG
CCAGATGGTAAGCCCTCCCGTATCGTAGTTATCTACACGACGGGGAGTC
AGGCAACTATGGATGAACGAAATAGACAGATCGCTGAGATAGGTGCCTC
ACTGATTAAGCATTGGTAACTGTCAGACCAAGTTTACTCATAACGCGTCA
ATTCGAGGGGGATCAATTCCGTGATAGGTGGGCTGCCCTTCCTGGTTGG
CTTGGTTTCATCAGCCATCCGCTTGCCCTCATCTGTTACGCCGGCGGTA
GCCGGCCAGCCTCGCAGAGCAGGATTCCCGTTGAGCACCGCCAGGTGC
GAATAAGGGACAGTGAAGAAGGAACACCCGCTCGCGGGTGGGCCTACT
TCACCTATCCTGCCCGGCTGACGCCGTTGGATACACCAAGGAAAGTCTA
CACGAACCCTTTGGCAAAATCCTGTATATCGTGCGAAAAAGGATGGATAT
ACCGAAAAAATCGCTATAATGACCCCGAAGCAGGGTTATGCAGCGGAAA
ACGGAATTGATCCGGCCACGATGCGTCCGGCGTAGAGGATCTGAAGAT
CAGCAGTTCAACCTGTTGATAGTACGTACTAAGCTCTCATGTTTCACGTA
CTAAGCTCTCATGTTTAACGTACTAAGCTCTCATGTTTAACGAACTAAACC
CTCATGGCTAACGTACTAAGCTCTCATGGCTAACGTACTAAGCTCTCATG
TTTCACGTACTAAGCTCTCATGTTTGAACAATAAAATTAATATAAATCAGC
AACTTAAATAGCCTCTAAGGTTTTAAGTTTTATAAGAAAAAAAAGAATATA
TAAGGCTTTTAAAGCTTTTAAGGTTTAACGGTTGTGGACAACAAGCCAGG
GATGTAACGCACTGAGAAGCCCTTAGAGCCTCTCAAAGCAATTTTGAGT
GACACAGGAACACTTAACGGCTGACATGGGAATTCCCCTCCACCGCGGT
GG

In this specific example, three plasmids were constructed which express a non-targeting control guide RNA (5′-TGATGGAGAGGTGCAAGTAG-3′, termed ‘NT’, SEQ ID NO:4), or guide RNAs targeting tdk_Bt (BT_2275) or susC_Bt (BT_3702) coding sequences on the Bt genome. The tdk gene encodes thymidine kinase, and the susC gene encodes an outer membrane protein in B. thetaiotaomicron involved in starch binding. The protospacer sequence for tdk_Bt is 5′-ATACAAGAGACCAGAAGAAG-3′(SEQ ID NO:5) and the protospacer sequence for susC_Bt is 5′-GCTCAAATCCGTATTCGTGG-3′ (SEQ ID NO: 6). In silico analyses of the non-targeting control protospacer sequence against Bacteroides genomes didn't result in any significant sequence matches, indicating that no ‘off-target’ activity. The targeting sequences for tdk_Bt and susC_Bt were selected to introduce a stop codon if C-to-T mutations occur at cytosine nucleotides (C) located approximately 15-20 bases upstream of the PAM (Nishida et a., Science, 2016, 353 (6305), doi: 10.1126/science.aaf8729; 12016, Banno et al., Nature Microbiology, 2018, 3. 10.1038/s41564-017-0102-6). The resulting plasmids are named pNBU2.CRISPR-CDA.NT, pNBU2.CRISPR-CDA.tdk_Bt and pNBU2.CRISPR-CDA.susC_Bt.

The pNBU2.CRISPR-CDA plasmids were conjugated to Bt cells with erythromycin selection, resulting in 500-1000 colonies per conjugation. Due to a lack of origin of replication for Bacteroides, these plasmids cannot be maintained. The erythromycin resistant colonies were likely chromosomal integrants. Colonies from each conjugation were picked for colony PCR screening of CRISPR-CDA integration at either one of the two attBT loci on the Bt chromosome. PCR using primers targeting chromosomal sequence at each attBT locus was used to deduce integration loci, followed by further junction PCR and DNA sequencing confirmation between chromosome and integration vector sequences. Three CRISPR-CDA integration strains with inducible CRISPR-CDA cassettes integrated at the attBT2-1 locus labeled NT (non-targeting), T (tdk_Bt) and S (susC_Bt) were obtained for the following inducible CRISPR base editing experiment. Single colonies of NT, T, and S CRISPR-CDA integrants were grown anaerobically in a coy chamber (Coy Laboratory Products Inc.) overnight in falcon tube cultures containing 5 ml TYG liquid medium (Holdeman et al., Anaerobe Laboratory Manual, 1977; Blacksburg, Va., Virginia Polytechnic Institute and State University Anaerobe Laboratory) supplemented with 200 μg/ml gentamicin (Gm) and 25 μg/ml erythromycin (Em). The cultures were diluted (10⁻⁶ or 10⁻⁸), and 100 μL were spread onto brain-heart infusion (BHI; Beckton Dickinson, Co.) blood agar plates (Gm 200 μg/mL and Em 25 μg/mL) supplemented with aTc at concentrations of 0 and 100 ng/ml, respectively. The agar plates were incubated anaerobically at 37° C. for 2-3 days. About 10²-10³ CFU (colony forming units) were obtained on each blood agar plate for all 3 strains.

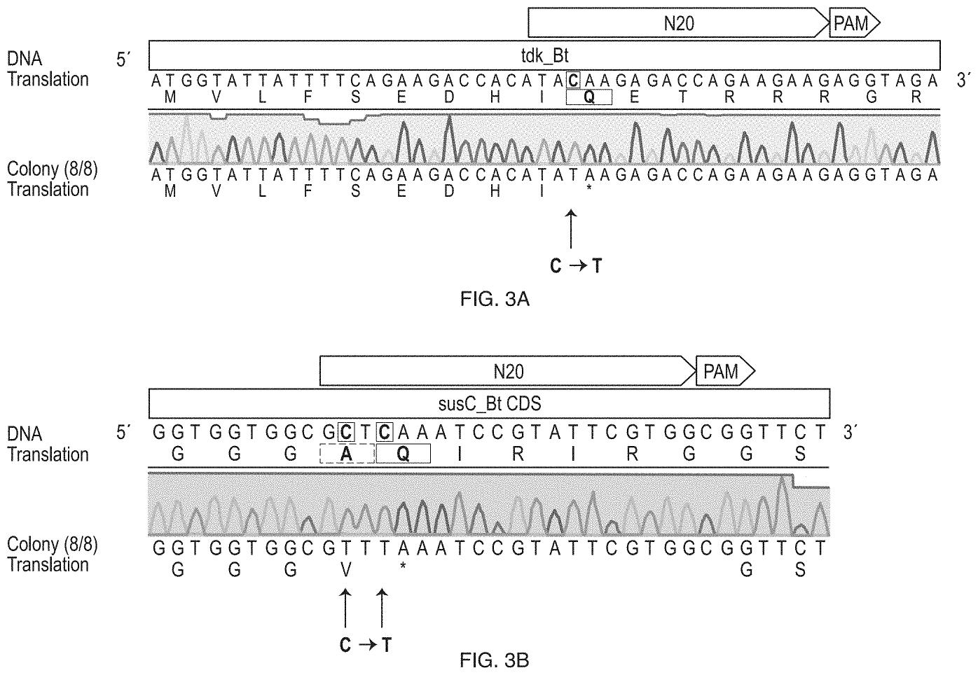

For tdk_Bt base editing, eight colonies were picked from aTc0 and aTc100 agar plates. These colonies were streaked on BHI blood agar plates supplemented with Gm at 200 μg/mL and 5-fluoro-20-deoxyuridine (FUdR) at 200 μg/mL, and incubated anaerobically at 37° C. for 2-3 days. While all colonies from aTc100 agar plate grew up, no growth was observed for colonies from aTc0agar plates. Colony PCR for the tdk_Bt region was performed followed by DNA sequencing. Sequencing results indicate eight out of eight colonies from the aTc100 agar plate harbors the expected C-to-T substitutions at the −17 position relative to the PAM, resulting in the introduction of an early stop codon (FIG. 3A). This tdk inactivation mutation confers resistance to the toxic nucleotide analog FUdR. Up to fifty colonies each from NT-aTc0, NT-aTc100, T-aTc0 and T-aTc100 agar plates were further streaked onto BHI blood agar plates supplemented with Gm at 200 μg/mL and FUdR at 200 μg/mL. It was observed that all colonies from T-aTc100 agar plates grew up while no growth was observed for other colonies. This suggests inducible, RNA guided, highly efficient nucleotide mutagenesis in Bt cells.

For susC_Bt base editing, eight colonies were picked from aTc0 and aTc100 agar plates. Colony PCR for the susC_Bt region was performed followed by DNA sequencing. Sequencing results indicate eight out of eight colonies from aTc100 agar plates harbor the expected C-to-T substitutions at the −17 and −19 positions relative to the PAM, resulting in an amino acid substitution (A to Vat position 491) and an early stop codon introduction (at position 493 of 3,012 bp susC coding sequence) (FIG. 3B). All eight colonies from aTc0 agar plate harbor the wild-type susC_Bt sequence. This indicates inducible, highly efficient, RNA guided base editing in Bt cells.

### Example 2. Stably Maintained CRISPR Base Editing in Bacteroides thetaiotaomicron VPI-5482

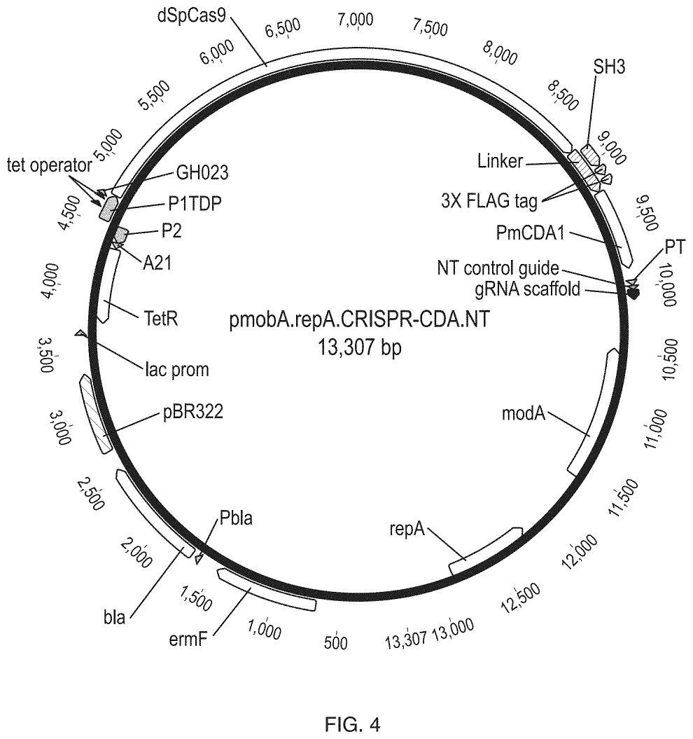

A Bacteroides dCas9-AID vector pmobA.repA.CRISPR-CDA.NT was constructed. The vector expresses (i) a catalytically inactivated Cas9 (dCas: D10A and H840A mutations) fused to Petromyzon marinus cytosine deaminase PmCDA1 (CDA) under an anhydrotetracycline-inducible promoter and (ii) a 20-nucleotide (nt) target sequence-gRNA scaffold hybrid (sgRNA) under a constitutive promoter P1. The plasmid contains a pBR322 origin of replication and bla sequence for ampicillin selection in E. coli. A mobA sequence is required for mobilization, a repA sequence for replication and an ermF sequence for erythromycin (Em) selection in Bacteroides (Smith, C. J., et al., Plasmid, 1995, 34, 211-222). The CRISPR-CDA unit consists of inducible, nuclease-deficient SpCas9 with D10A and H840A mutations fused with Petromyzon marinus cytosine deaminase (PmCDA1). The dCas9-CDA1 fusion was controlled by TetR regulator (P2-A21-tetR, P1TDP-GH023-dSpCas9-PmCDA1) under the control of anhydrotetracycline (aTc), and the guide RNA was controlled by constitutive P1 promoter (P1-N20 sgRNA scaffold). The promoters and ribosomal binding sites are derived and engineered from regulatory sequences of Bacteroides thetaiotaomicron (Bt) 16S rRNA genes, as described in Lim et al., Cell, 2017, 169:547-558. The guide RNA is a nucleotide sequence that is homologous to a coding or non-coding DNA sequence or is a non-targeting scramble nucleotide sequence. This sequence can vary as long as it is compatible with protospacer adjacent motif (PAM) requirements of different Cas9 homologs. The guide RNA can be either in separate transcriptional units of tracrRNA and crRNA or fused into a hybrid chimeric tracr/crRNA single guide (sgRNA). A map of plasmid pmobA.repA.CRISPR-CDA.NT DNA sequence (13,307 bp) is shown in FIG. 4 and listed as SEQ ID NO: 7:

TCGGGACGCTCATCAATATCCACCCTGCCTGGGATAAATCCTCGCCCTG
CATTTTTAGAACCACGTTTGGCATACCTGCGACCTTGTCTGCGAAGATAT
TTGTGCAGTTTGCCACCCCGCCGCTTATCCTCCCAAATCCAGCGATATAT
CGTTTCGTGAGATACCATCGCAATTCCCTCCAAGCGGCTCCTGCCGACA
ATCTGCTCCGGGCTGAATCCTTTCTTCAACAGCTTTATTATCCGTTTTCTC
ATTGCCGGTGTAAGCACTTCCTTGCGATGTTTTTGCTGCTTGCGCCTGTC
TGCTTTTCGCTGGGCAAGCTCCATGCTATAGCTACCACTTCGGGCGTCG
CAATTGCGCTTTATCTCCCTGTAAACAGTGCTTTTATCTACTCCGATAGCT
TCCGCTATTGCTTTTTTGCTCATCGGTATTTGCAACATCATAGAAATTGCA
TACCTTTGTTCCTCGGTTATATGTTTGCTCATCTGCAACTTTTTTTTCTTT
GGACGGACAATTAAAGCAAAGATAGCAAACTTTATCCATTCAGAGTGAGAG
AAAGGGGGACATTGTCTCTCTTTCCTCTCTGAAAAATAAATGTTTTTATTG
CTTATTATCCGCACCCAAAAAGTTGCATTTATAAGTTGAACTCAAGAAGTA
TTCACCTGTAAGAAGTTACTAATGACAAAAAAGAAATTGCCCGTTCGTTTT
ACGGGTCAGCACTTTACTATTGATAAAGTGCTAATAAAAGATGCAATAAG
ACAAGCAAATATAAGTAATCAGGATACGGTTTTAGATATTGGGGCAGGCA
AGGGGTTTCTTACTGTTCATTTATTAAAAATCGCCAACAATGTTGTTGCTA
TTGAAAACGACACAGCTTTGGTTGAACATTTACGAAAATTATTTTCTGATG
CCCGAAATGTTCAAGTTGTCGGTTGTGATTTTAGGAATTTTGCAGTTCCG
AAATTTCCTTTCAAAGTGGTGTCAAATATTCCTTATGGCATTACTTCCGAT
ATTTTCAAAATCCTGATGTTTGAGAGTCTTGGAAATTTTCTGGGAGGTTC
CATTGTCCTTCAATTAGAACCTACACAAAAGTTATTTTCGAGGAAGCTTTA
CAATCCATATACCGTTTTCTATCATACTTTTTTTGATTTGAAACTTGTCTA
TGAGGTAGGTCCTGAAAGTTTCTTGCCACCGCCAACTGTCAAATCAGCCC
TGTTAAACATTAAAAGAAAACACTTATTTTTTGATTTTAAGTTTAAAGCCA
AATACTTAGCATTTATTTCCTGTCTGTTAGAGAAACCTGATTTATCTGTAA
AAACAGCTTTAAAGTCGATTTTCAGGAAAAGTCAGGTCAGGTCAATTTCGG
AAAAATTCGGTTTAAACCTTAATGCTCAAATTGTTTGTTTGTCTCCAAGTC
AATGGTTAAACTGTTTTTTGGAAATGCTGGAAGTTGTCCCTGAAAAATTTC
ATCCTTCGTAGTTCAAAGTCGGGTGGTTGTCAAGATGATTTTTTTGGTTT
GGTGTCGTCTTTTTTTAAGCTGCCGCATAACGGCTGGCAAATTGGCGAT
GGAGCCGACTTTGGTGGCACTTTTCGGGGAAATGTGCGCGGAACCCCT
ATTTGTTTATTTTTCTAAATACATTCAAATATGTATCCGCTCATGAGACAA
TAACCCTGATAAATGCTTCAATAATATTGAAAAAGGAAGAGTATGAGTATT
CAACATTTCCGTGTCGCCCTTATTCCCTTTTTTGCGGCATTTTGCCTTCCT
GTTTTTGCTCACCCAGAAACGCTGGTGAAAGTAAAAGATGCTGAAGATCAG
TTGGGTGCACGAGTGGGTTACATCGAACTGGATCTCAACAGCGGTAAGA
TCCTTGAGAGTTTTCGCCCCGAAGAACGTTTTCCAATGATGAGCACTTTT
AAAGTTCTGCTATGTGGCGCGGTATTATCCCGTATTGACGCCGGGCAAG
AGCAACTCGGTCGCCGCATACACTATTCTCAGAATGACTTGGTTGAGTAC
TCACCAGTCACAGAAAAGCATCTTACGGATGGCATGACAGTAAGAGAATT
ATGCAGTGCTGCCATAACCATGAGTGATAACACTGCGGCCAACTTACTTC
TGACAACGATCGGAGGACCGAAGGAGCTAACCGCTTTTTTGCACAACAT
GGGGGATCATGTAACTCGCCTTGATCGTTGGGAACCGGAGCTGAATGAA
GCCATACCAAACGACGAGCGTGACACCACGATGCCTGTAGCAATGGCAA
CAACGTTGCGCAAACTATTAACTGGCGAACTACTTACTCTAGCTTCCCGG
CAACAATTAATAGACTGGATGGAGGCGGATAAAGTTGCAGGACCACTTC
TGCGCTCGGCCCTTCCGGCTGGCTGGTTTATTGCTGATAAATCTGGAGC
CGGTGAGCGTGGGTCTCGCGGTATCATTGCAGCACTGGGGCCAGATGG
TAAGCCCTCCCGTATCGTAGTTATCTACACGACGGGGAGTCAGGCAACT
ATGGATGAACGAAATAGACAGATCGCTGAGATAGGTGCCTCACTGATTA
AGCATTGGTAACTGTCAGACCAAGTTTACTCATATATACTTTAGATTGATT
TAAAACTTCATTTTTAATTTAAAAGGATCTAGGTGAAGATCCTTTTTGATA
ATCTCATGACCAAAATCCCTTAACGTGAGTTTTCGTTCCACTGAGCGTCAG
ACCCCGTAGAAAAGATCAAAGGATCTTCTTGAGATCCTTTTTTCTGCGCG
TAATCTGCTGCTTGCAAACAAAAAAACCACCGCTACCAGCGGTGGTTTGT
TTGCCGGATCAAGAGCTACCAACTCTTTTTCCGAAGGTAACTGGCTTCAG
CAGAGCGCAGATACCAAATACTGTTCTTCTAGTGTAGCCGTAGTTAGGC
CACCACTTCAAGAACTCTGTAGCACCGCCTACATACCTCGCTCTGCTAAT
CCTGTTACCAGTGGCTGCTGCCAGTGGCGATAAGTCGTGTCTTACCGGG
TTGGACTCAAGACGATAGTTACCGGATAAGGCGCAGCGGTCGGGCTGA
ACGGGGGGTTCGTGCACACAGCCCAGCTTGGAGCGAACGACCTACACC
GAACTGAGATACCTACAGCGTGAGCTATGAGAAAGCGCCACGCTTCCCG
AAGGGAGAAAGGCGGACAGGTATCCGGTAAGCGGCAGGGTCGGAACAG
GAGAGCGCACGAGGGAGCTTCCAGGGGGAAACGCCTGGTATCTTTATA
GTCCTGTCGGGTTTCGCCACCTCTGACTTGAGCGTCGATTTTTGTGATGC
TCGTCAGGGGGGCGGAGCCTATGGAAAAACGCCAGCAACGCGGCCTTT
TTACGGTTCCTGGCCTTTTGCTGGCCTTTTGCTCACATGTTCTTTCCTGC
GTTATCCCCTGATTCTGTGGATAACCGTATTACCGCCTTTGAGTGAGCTG
ATACCGCTCGCCGCAGCCGAACGACCGAGCGCAGCGAGTCAGTGAGCG
AGGAAGCGGAAGAGCGCCCAATACGCAAACCGCCTCTCCCCGCGCGTT
GGCCGATTCATTAATGCAGCTGGCACGACAGGTTTCCCGACTGGAAAGC
GGGCAGTGAGCGCAACGCAATTAATGTGAGTTAGCTCACTCATTAGGCA
CCCCAGGCTTTACACTTTATGCTTCCGGCTCGTATGTTGTGTGGAATTGT
GAGCGGATAACAATTTCACACAGGAAACAGCTATGACCATGATTACGCC
CTTAAGACCCACTTTCACATTTAAGTTGTTTTTCTAATCCGCATATGATCA
ATTCAAGGCCGAATAAGAAGGCTGGCTCTGCACCTTGGTGATCAAATAAT
TCGATAGCTTGTCGTAATAATGGCGGCATACTATCAGTAGTAGGTGTTTC
CCTTTCTTCTTTAGCGACTTGATGCTCTTGATCTTCCAATACGCAACCTAA
AGTAAAATGCCCCACAGCGCTGAGTGCATATAATGCATTCTCTAGTGAAA
AACCTTGTTGGCATAAAAAGGCTAATTGATTTTCGAGAGTTTCATACTGTT
TTTCTGTAGGCCGTGTACCTAAATGTACTTTTGCTCCATCGCGATGACTT
AGTAAAGCACATCTAAAACTTTTAGCGTTATTACGTAAAAAATCTTGCCAG
CTTTCCCCTTCTAAAGGGCAAAAGTGAGTATGGTGCCTATCTAACATCTC
AATGGCTAAGGCGTCGAGCAAAGCCCGCTTATTTTTTACATGCCAATACA
ATGTAGGCTGCTCTACACCTAGCTTCTGGGCGAGTTTACGGGTTGTTAAA
CCTTCGATTCCGACCTCATTAAGCAGCTCTAATGCGCTGTTAATCACTTT
ACTTTTATCTAATCTAGACATATTCGTTTAATATCATAAATAATTTATTTT
ATTTTAAAATGCGCGGGTGCAAAGGTAAGAGGTTTTATTTTAACTACCAAA
TGTTTTCGGAAGTTTTTTCGCTTTTCTTTTTCTATCGTTTCTCAGACTCTC
TTAGCGAAAGGGAAAGAAGGTAAAGAAGAAAAACAAAACGCCTTTTCTTTT
TTGCACCCGCTTTCCAAGAGAAGAAAGCCTTGTTAAATTGACTTAGTGTAA
AAGCGCAGTACTGCTTGACCATAAGAACAAAAAAATCTCTATCACTGATA
GGGATAAAGTTTGGAAGATAAAGCTAAAAGTTCTTATCTTTGCAGTCTCC
CTATCAGTGATAGAGACGAAATAAAGACATATAAAAGAAAAGACACCATG
GATAAGAAATACTCAATAGGCTTAGCTATCGGCACAAATAGCGTCGGATG
GGCGGTGATCACTGATGAATATAAGGTTCCGTCTAAAAAGTTCAAGGTTC
TGGGAAATACAGACCGCCACAGTATCAAAAAAAATCTTATAGGGGCTCTT
TTATTTGACAGTGGAGAGACAGCGGAAGCGACTCGTCTCAAACGGACAG
CTCGTAGAAGGTATACACGTCGGAAGAATCGTATTTGTTATCTACAGGAG
ATTTTTTCAAATGAGATGGCGAAAGTAGATGATAGTTTCTTTCATCGACTT
GAAGAGTCTTTTTTGGTGGAAGAAGACAAGAAGCATGAACGTCATCCTAT
TTTTGGAAATATAGTAGATGAAGTTGCTTATCATGAGAAATATCCAACTAT
CTATCATCTGCGAAAAAAATTGGTAGATTCTACTGATAAAGCGGATTTGC
GCTTAATCTATTTGGCCTTAGCGCATATGATTAAGTTTCGTGGTCATTTTT
TGATTGAGGGAGATTTAAATCCTGATAATAGTGATGTGGACAAACTATTT
ATCCAGTTGGTACAAACCTACAATCAATTATTTGAAGAAAACCCTATTAAC
GCAAGTGGAGTAGATGCTAAAGCGATTCTTTCTGCACGATTGAGTAAATC
AAGACGATTAGAAAATCTCATTGCTCAGCTCCCCGGTGAGAAGAAAAATG
GCTTATTTGGGAATCTCATTGCTTTGTCATTGGGTTTGACCCCTAATTTTA
AATCAAATTTTGATTTGGCAGAAGATGCTAAATTACAGCTTTCAAAAGATA
CTTACGATGATGATTTAGATAATTTATTGGCGCAAATTGGAGATCAATATG
CTGATTTGTTTTTGGCAGCTAAGAATTTATCAGATGCTATTTTACTTTCAG
ATATCCTAAGAGTAAATACTGAAATAACTAAGGCTCCCCTATCAGCTTCA
ATGATTAAACGCTACGATGAACATCATCAAGACTTGACTCTTTTAAAAGCT
TTAGTTCGACAACAACTTCCAGAAAAGTATAAAGAAATCTTTTTTGATCAA
TCAAAAAACGGATATGCAGGTTATATTGATGGGGGAGCTAGCCAAGAAG
AATTTTATAAATTTATCAAACCAATTTTAGAAAAAATGGATGGTACTGAGG
AATTATTGGTGAAACTAAATCGTGAAGATTTGCTGCGCAAGCAACGGACC
TTTGACAACGGCTCTATTCCCCATCAAATTCACTTGGGTGAGCTGCATGC
TATTTTGAGAAGACAAGAAGACTTTTATCCATTTTTAAAAGACAATCGTGA
GAAGATTGAAAAAATCTTGACTTTTCGAATTCCTTATTATGTTGGTCCATT
GGCGCGTGGCAATAGTCGTTTTGCATGGATGACTCGGAAGTCTGAAGAA
ACAATTACCCCATGGAATTTTGAAGAAGTTGTCGATAAAGGTGCTTCAGC
TCAATCATTTATTGAACGCATGACAAACTTTGATAAAAATCTTCCAAATGA
AAAAGTACTACCAAAACATAGTTTGCTTTATGAGTATTTTACGGTTTATAA
CGAATTGACAAAGGTCAAATATGTTACTGAAGGAATGCGAAAACCAGCAT
TTCTTTCAGGTGAACAGAAGAAAGCCATTGTTGATTTACTCTTCAAAACAA
ATCGAAAAGTAACCGTTAAGCAATTAAAAGAAGATTATTTCAAAAAAATAG
AATGTTTTGATAGTGTTGAAATTTCAGGAGTTGAAGATAGATTTAATGCTT
CATTAGGTACCTACCATGATTTGCTAAAAATTATTAAAGATAAAGATTTTT
TGGATAATGAAGAAAATGAAGATATCTTAGAGGATATTGTTTTAACATTGA
CCTTATTTGAAGATAGGGAGATGATTGAGGAAAGACTTAAAACATATGCTC
ACCTCTTTGATGATAAGGTGATGAAACAGCTTAAACGTCGCCGTTATACT
GGTTGGGGACGTTTGTCTCGAAAATTGATTAATGGTATTAGGGATAAGCA
ATCTGGCAAAACAATATTAGATTTTTTGAAATCAGATGGTTTTGCCAATCG
CAATTTTATGCAGCTGATCCATGATGATAGTTTGACATTTAAAGAAGACAT
TCAAAAAGCACAAGTGTCTGGACAAGGCGATAGTTTACATGAACATATTG
CAAATTTAGCTGGTAGCCCTGCTATTAAAAAAGGTATTTTACAGACTGTAA
AAGTTGTTGATGAATTGGTCAAAGTAATGGGGCGGCATAAGCCAGAAAA
TATCGTTATTGAAATGGCACGTGAAAATCAGACAACTCAAAAGGGCCAGA
AAAATTCGCGAGAGCGTATGAAACGAATCGAAGAAGGTATCAAAGAATTA
GGAAGTCAGATTCTTAAAGAGCATCCTGTTGAAAATACTCAATTGCAAAA
TGAAAAGCTCTATCTCTATTATCTCCAAAATGGAAGAGACATGTATGTGG
ACCAAGAATTAGATATTAATCGTTTAAGTGATTATGATGTCGATGCCATTG
TTCCACAAAGTTTCCTTAAAGACGATTCAATAGACAATAAGGTCTTAACG
CGTTCTGATAAAAATCGTGGTAAATCGGATAACGTTCCAAGTGAAGAAGT
AGTCAAAAAGATGAAAAACTATTGGAGACAACTTCTAAACGCCAAGTTAA
TCACTCAACGTAAGTTTGATAATTTAACGAAAGCTGAACGTGGAGGTTTG
AGTGAACTTGATAAAGCTGGTTTTATCAAACGCCAATTGGTTGAAACTCG
CCAAATCACTAAGCATGTGGCACAAATTTTGGATAGTCGCATGAATACTA
AATACGATGAAAATGATAAACTTATTCGAGAGGTTAAAGTGATTACCTTAA
AATCTAAATTAGTTTCTGACTTCCGAAAAGATTTCCAATTCTATAAAGTAC
GTGAGATTAACAATTACCATCATGCCCATGATGCGTATCTAAATGCCGTC
GTTGGAACTGCTTTGATTAAGAAATATCCAAAACTTGAATCGGAGTTTGT
CTATGGTGATTATAAAGTTTATGATGTTCGTAAAATGATTGCTAAGTCTGA
GCAAGAAATAGGCAAAGCAACCGCAAAATATTTCTTTTACTCTAATATCAT
GAACTTCTTCAAAACAGAAATTACACTTGCAAATGGAGAGATTCGCAAAC
GCCCTCTAATCGAAACTAATGGGGAAACTGGAGAAATTGTCTGGGATAA
AGGGCGAGATTTTGCCACAGTGCGCAAAGTATTGTCCATGCCCCAAGTC
AATATTGTCAAGAAAACAGAAGTACAGACAGGCGGATTCTCCAAGGAGT
CAATTTTACCAAAAAGAAATTCGGACAAGCTTATTGCTCGTAAAAAAGACT
GGGATCCAAAAAAATATGGTGGTTTTGATAGTCCAACGGTAGCTTATTCA
GTCCTAGTGGTTGCTAAGGTGGAAAAAGGGAAATCGAAGAAGTTAAAAT
CCGTTAAAGAGTTACTAGGGATCACAATTATGGAAAGAAGTTCCTTTGAA
AAAAATCCGATTGACTTTTTAGAAGCTAAAGGATATAAGGAAGTTAAAAAA
GACTTAATCATTAAACTACCTAAATATAGTCTTTTTGAGTTAGAAAACGGT
CGTAAACGGATGCTGGCTAGTGCCGGAGAATTACAAAAAGGAAATGAGC
TGGCTCTGCCAAGCAAATATGTGAATTTTTTATATTTAGCTAGTCATTATG
AAAAGTTGAAGGGTAGTCCAGAAGATAACGAACAAAAACAATTGTTTGTG
GAGCAGCATAAGCATTATTTAGATGAGATTATTGAGCAAATCAGTGAATT
TTCTAAGCGTGTTATTTTAGCAGATGCCAATTTAGATAAAGTTCTTAGTGC
ATATAACAAACATAGAGACAAACCAATACGTGAACAAGCAGAAAATATTA
TTCATTTATTTACGTTGACGAATCTTGGAGCTCCCGCTGCTTTTAAATATT
TTGATACAACAATTGATCGTAAACGATATACGTCTACAAAAGAAGTTTTAG
ATGCCACTCTTATCCATCAATCCATCACTGGTCTTTATGAAACACGCATT
GATTTGAGTCAGCTAGGAGGTGACGGTGGAGGAGGTTCTGGAGGTGGA
GGTTCTGCTGAGTATGTGCGAGCCCTCTTTGACTTTAATGGGAATGATGA
AGAGGATCTTCCCTTTAAGAAAGGAGACATCCTGAGAATCCGGGATAAG
CCTGAGGAGCAGTGGTGGAATGCAGAGGACAGCGAAGGAAAGAGGGG
GATGATTCCTGTCCCTTACGTGGAGAAGTATTCCGGAGACTATAAGGAC
CACGACGGAGACTACAAGGATCATGATATTGATTACAAAGACGATGACG
ATAAGTCTAGGCTCGAGTCCGGAGACTATAAGGACCACGACGGAGACTA
CAAGGATCATGATATTGATTACAAAGACGATGACGATAAGTCTAGGATGA
CCGACGCTGAGTACGTGAGAATCCATGAGAAGTTGGACATCTACACGTT
TAAGAAACAGTTTTTCAACAACAAAAAATCCGTGTCGCATAGATGCTACG
TTCTCTTTGAATTAAAACGACGGGGTGAACGTAGAGCGTGTTTTTGGGG
CTATGCTGTGAATAAACCACAGAGCGGGACAGAACGTGGCATTCACGCC
GAAATCTTTAGCATTAGAAAAGTCGAAGAATACCTGCGCGACAACCCCG
GACAATTCACGATAAATTGGTACTCATCCTGGAGTCCTTGTGCAGATTGC
GCTGAAAAGATCTTAGAATGGTATAACCAGGAGCTGCGGGGGAACGGC
CACACTTTGAAAATCTGGGCTTGCAAACTCTATTACGAGAAAAATGCGAG
GAATCAAATTGGGCTGTGGAATCTCAGAGATAACGGGGTTGGGTTGAAT
GTAATGGTAAGTGAACACTACCAATGTTGCAGGAAAATATTCATCCAATC
GTCGCACAATCAATTGAATGAGAATAGATGGCTTGAGAAGACTTTGAAGC
GAGCTGAAAAACGACGGAGCGAGTTGTCCATTATGATTCAGGTAAAAATA
CTCCACACCACTAAGAGTCCTGCTGTTTAAATTAATGCGGCTGCAATTTT
TTTGGGCGGGGCCGCCCAAAAAAATCCTAGCACCCTGCAGCAGTACTGC
TTGACCATAAGAACAAAAAAACTTCCGATAAAGTTTGGAAGATAAAGCTA
AAAGTTCTTATCTTTGCAGTTGATGGAGAGGTGCAAGTAGGTTTTAGAGC
TAGAAATAGCAAGTTAAAATAAGGCTAGTCCGTTATCAACTTGAAAAAGT
GGCACCGAGTCGGTGCTTTTTTTGTCGACTCTAGAGGATCCCCGGGTAC
CGAGCTCGAATTCACTGGCCGTCGTTTTACAACGTCGTGACTGGGAAAA
CCCTGGCGTTACCCAACTTAATCGCCTTGCAGCACATCCCCCTTTCGCC
AGCTGGCGTAATAGCGAAGAGGCCCGCACCGATCGCCCTTCCCAACAG
TTGCGCAGCCTGAATGGCGAATGGCGCCTGATGCGGTATTTTCTCCTTA
CGCATCTGTGCGGTATTTCACACCGCATACACACCATAAACTTTTTTTAG
AATAAGCACACAACCGTTTTCCGAACCCTGCAAAATGTTTTCTGAATCCG
AACGGTGTAACACTCCATTGAGAGAGGCTGCCGTTTGGTCGCTCCCCCT
TTGGGGGCGGGGGGGGGTTACATACCCATGCCGAAACCTCTGCTTCTG
GTGATTTGCTTGAATAGGTCTTTCCCCTCTTCCATAGCTTTTGATATGTTT
GGGAAATGATGCCTTAAAGCCTCCAGTTGTTCGGAATTGAACAAGTCTTT
CATCTTACCAAGTTCTTTTTTCAACTCCTTGGTTTCGGCTTTTAGTTTTTG
GTTCTCCGTCCTTAATAGGTTACTGGTTGTCCTTGCGTTGTCCATTTGTT
GTCTATAATACTCCTTGTCATTCTCGGCTTTGAATGCCTTTGTGCTGTTTC
GCTCTTTTTCAAGTATAGCCTTTCCCAGTCTATCGGATAGTTGTTCATTTT
CCCCCTCTAAAGTCTTTACTTTGGCTTTTAAGGCATCCTTTTCCCTATCGT
TGACTGTTTTTCCAATCAAGCCGTAAAACTTCTCTGAAGCCTTAGAAATG
AGTTTTTGGACGTTCTTCTTTGTTTCAATGGAACGTAGTTCCTTCTGAAGC
TGAAGAAGCTGGTTTTGTGCGTCCTTGTATTTGTCTAATGCACTGGATAT
ATCGTTGGATAGTTCCTGAAGCTGTTCTTTCGCACATTCGGTCTTGTACT
GCATAGCCGATAAGTGTTTGCGGTCAGAAGAAACGCCACGTTCCATGCC
CAGTGTTTCAGATGCTATGGTTTGGAGTTCTGCCATGTCATCACGCGATA
AACGCACACTTTTCCCATTCGGCTGCGTCCAATCGAAAACTACATGGGC
ATGAAGGTTAGGTGTCCACTGCTTTGCGTTCATGTATCCTTCGTCCTTGT
GTATATGGATTTGAAACGCTTCGATACCGAAACGTTCTTTGCAGACCGTG
GCAAACTGCTGGAGTTCCTGCATAGTGGTTTCTTGTTTGATTACTATTACT
CCCTCTCGTATGGGTGCGGCTTTAGCCTGCATCTTCTGCCCAACCGTAT
CGAGATATCTTTGTTTTGCACTCTCCAGCCGATGGGAAATGCTATCTCCA
ACCCAGCTTTCATTCAAATGACTAAGTTCGGGACGAACATAGTCCAACTC
TTTTTCCCTAAAGTTGTGAATCTCGCTCCCCGGCTTCACTGCTTGTACAT
GAATACTTGTTGCTCCCATAAGTTAACATTTTTGTGACAATCGATAACAGC
CGGTGACAGCCGGCTGACAGGGGGTTAAGGGGGCTTGTCCCCTTACAC
ACGCACTCTTTAGGGTGCTAGTGTGCTATCACCATACTGCATAGGTGCG
AAGTTAGTGAATGTTTTGTAAATGCACAAATAAAGGGAAAAACATTTGGAT
TTGCGATAATAAAGTACTACCTTTGTTGCTGACCAAACGGTAGCTGACCG
ATACGGGAGAGTTACCAAAATACAAGCCGCTGGAGTTAATTGACGGACA
TCCGACATCTCCAGCGGCTTTATTTTTGCCTATCTGCTTCGCCTAGGCAC
ACCAGTACCTCTACTAAAAATGTACTTCAAAGATACTTATTTTCTACCGAC
TTGATAGTTTTTACCCCATATTCTTGGACATTTTTCCCCCATGAGGTTATC
TTTGTAGGGTGAAAGAGAAACCCATAAACGGGGATAGATTGAATGCTGG
GAAGCATAAACAATCGGGGTAAGGTTAGCGAACCTTGCCTTTCATCCCC
CATTATAACTTTACATAGAGGAACTTTATCTATCCCCCCCCGCCCCCAAA
GGGGGAGCGACCAAACGGCAGCTTCACTCAATGGAGTGTTACTGTTCAT
CAAAGCCAAGTGATAATTGTCGTTTCTCTGCTTCTTCTTTCTTTTGGGCAG
CTAAAGTCTTTTTCCGAACGTATGTTTTAGCAAATGTCACTCGGTCACCAT
TGAATACTATCAGAGGATTAATAAACCAAAGATTATCGGCTGGTCCTCGG
GCTATGATTTCAGCTTTTACAAGTTCTGCAAGTCCTTTATAAACGGCTTTG
TCTGTTTTGTATTTGGTATATTCTAGGCATTTTTTTCTATTGAAAATGATT
AAATCATTTTTGGGTTTCATGCAGGTCATAAAGTAACCAAAAACCCGAATA
GCTGCTTGTGATAGGTCAAAGAATGCAGCAAAGTTAGAAAGATACAATTT
AGTGAATTGTTCTTCATCTACTTCTATTTGACGGATAAACGAAGTCTTAAA
CACTTCTCCAGTTTCAGTGTCGGCTAAAGCTACTACAGCTCTCTTATCGC
CACCACTATTACTCTTATACTTTTTAACAACATGATTTTCAATACCTTCTA
TAGCTTGTTTCATAAAAGGATTTTCTTCGTTCTTTTGAAAATCGGTTAACT
TAACTGCTTTTTTATTTTCCATTTTGATATGTTTTTGGGAAATATTATTCT
CCACAAAGTAAACTATTATTTTCCATAAAAACAATATTAAGGGAAATATTA
TTTTCCTATTTAGTATCATATTAGGAAATCGGTATTTTCTAGATTGGAAAA
TGAGAATTTCCAATATGGAAAATGCCCTATATTGTGTATCAAGTACTTAAC
TTATTCTATTTCTTTTATTCTTAATATACCCCCAAAACAGCACAAAATCAG
TCACTTAAAAATCATCGGTCGGGGAATGGTGCACTCTCAGTACAATCTGCT
CTGATGCCGCATAGTTAAGCCAGCCCCGACACCCGCCAACACCCGCTGAC
GCGCCCTGACGGGCTTGTCTGCTCCCGGCATCCGCTTACAGACAAGCT
GTGACCGTCTCCGGGAGCTGCATGTGTCAGAGGTTTTCACCGTCATCAC
CGAAACGCGCGAGACGAAAGGGCCTCGTGATACGCCTATTTTTATAGGT
TAATGTCATGATAATAATGGTTTCTTAGCTAAATTTAAATATAAACAA

In this specific example, three plasmids were constructed which express a non-targeting control guide RNA (5′-TGATGGAGAGGTGCAAGTAG-3′ termed ‘NT’, SEQ ID NO: 4), or a guide RNA targeting BT_0362 or BT_0364 coding sequences on the Bt genome. The protospacer sequence for BT_0362 is 5′-GGACGAATCGTAAATGCAGA-3′ (SEQ ID NO: 8) and the protospacer sequence for BT_0364 is 5′-CCCATTGGCTGAATGTGGCG-3′ (SEQ ID NO: 9). In silico analyses of the non-targeting control protospacer sequence against Bacteroides genomes didn't result in any significant sequence matches, indicating no ‘off-target’ activity. The targeting sequences for BT_0362 and BT_0364 were selected to introduce a stop codon if C-to-T mutations occur at cytosine nucleotides (C) located approximately 15-20 bases upstream of the PAM (Nishida et a., Science, 2016, 353 (6305), doi:10.1126/science.aaf8729; 12016, Banno et al., Nature Microbiology, 2018, 3.10.1038/s41564-017-0102-6). The resulting plasmids are named pmobA.repA.CRISPR-CDA.NT, pmobA.repA.CRISPR-CDA.BT_0362 and pmobA.repA.CRISPR-CDA.BT_0364.

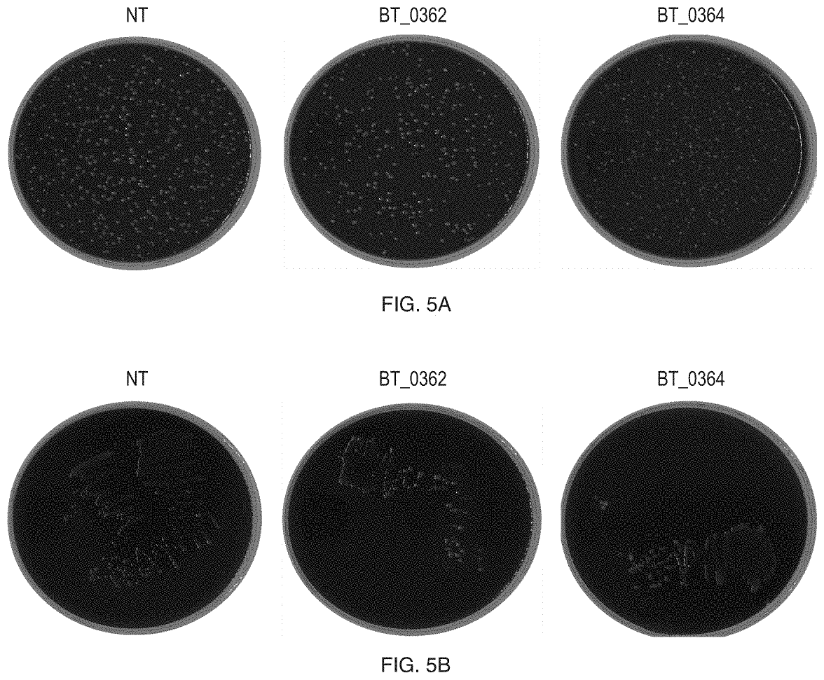

The pmobA.repA.CRISPR-CDA plasmids were conjugated into Bt cells initially under no selection or induction on brain-heart infusion (BHI; Beckton Dickinson, Co.) blood agar plates under aerobic conditions. This conjugation smear was scraped off and reconstituted with 1 ml of TYG liquid medium (Holdeman et al., Anaerobe Laboratory Manual, 1977; Blacksburg, Va., Virginia Polytechnic Institute and State University Anaerobe Laboratory). For each conjugated plasmid sample in TYG medium, 100 μl of a 1:10 dilution in TYG medium was plated on 25 μg/ml erythromycin (Em) and 200 μg/ml gentamicin (Gm) BHI 10% blood agar plates, resulting in hundreds of colonies per conjugation (FIG. 5A). Due to the repA origin of replication for Bacteroides, these plasmids can be maintained. Single colonies from each conjugation were picked for continued TYG medium liquid culture growth under 25 μg/ml erythromycin (Em) and 200 μg/ml gentamicin (Gm) selection followed by plasmid purification to verify correct plasmid maintenance. PCR amplification and Sanger sequencing of the pmobA.repA.CRISPR-CDA guide region verified the correct guide sequence for each plasmid. Three pmobA.repA.CRISPR-CDA stably maintained plasmid strains labeled NT (nontargeting), BT_0362 and BT_0364 were obtained for the following inducible CRISPR base editing experiment. Single colonies of NT, BT_0362, and BT_0364 pmobA.repA.CRISPR-CDA plasmid strains were grown anaerobically in a coy chamber (Coy Laboratory Products Inc.) overnight in falcon tube cultures containing 5 ml TYG liquid medium supplemented with 200 μg/ml gentamicin (Gm), 25 μg/ml erythromycin (Em) and 100 ng/ml aTc. Samples from these cultures were then streaked with a plastic loop onto BHI 10% blood agar plates (Gm 200 μg/mL and Em 25 μg/mL) supplemented with aTc at 100 ng/ml. The agar plates were incubated anaerobically at 37° C. for 2-3 days. Individual colonies were obtained along the loop streak areas on each blood agar plate for all 3 strains (FIG. 5B).

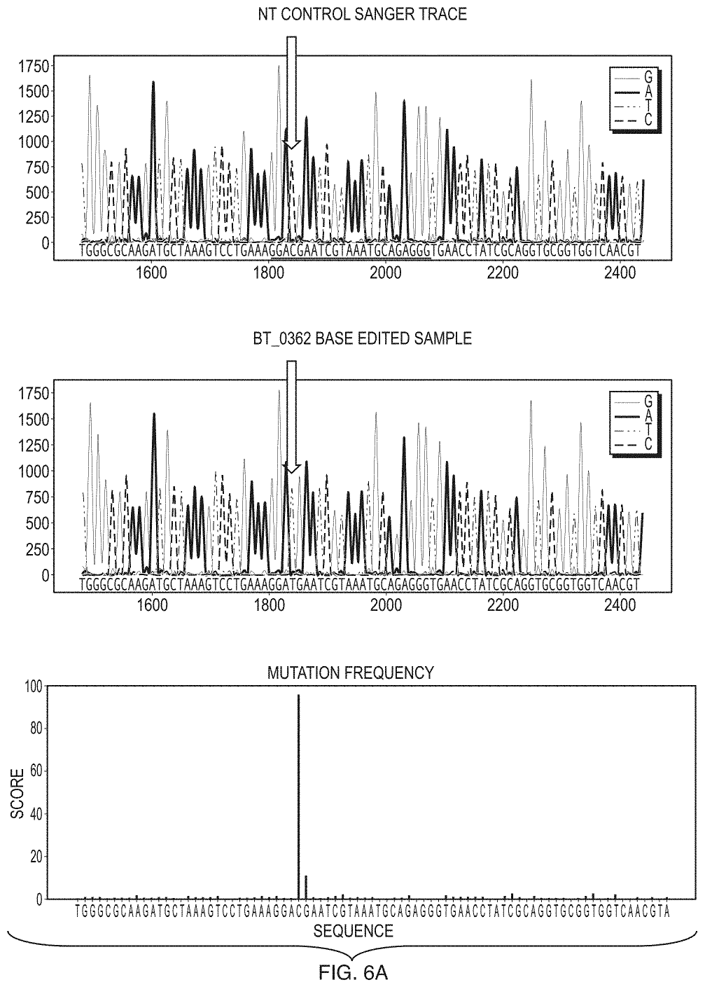

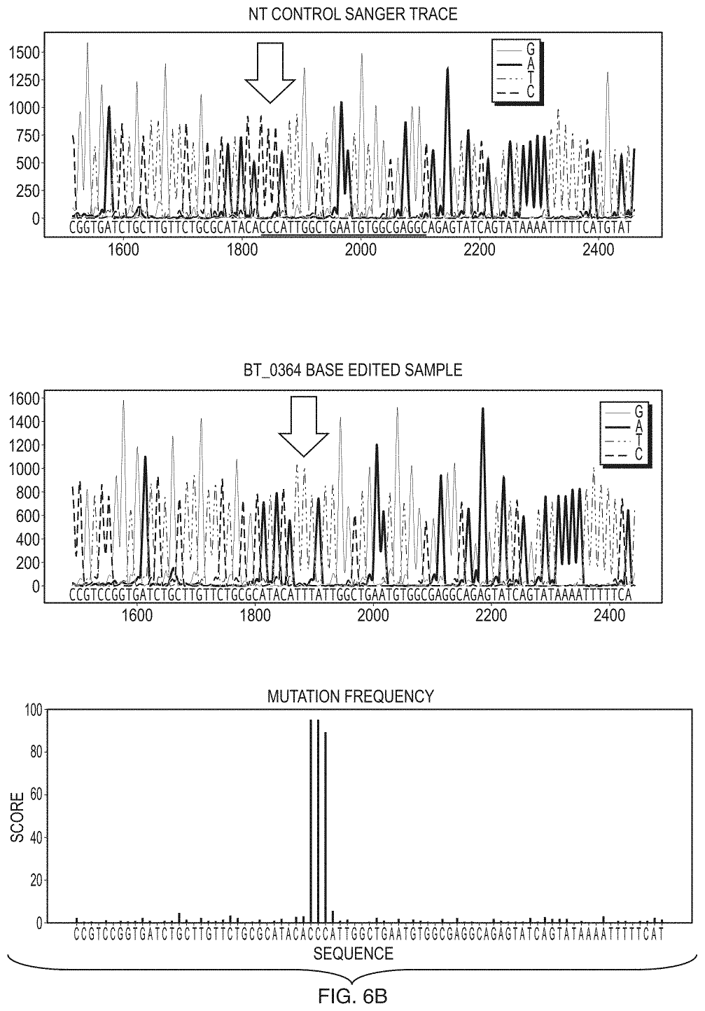

Colonies were picked from these three aTc100 agar plates. Colony PCR for the BT_0362 and BT_0364 region was performed followed by Sanger sequencing. Quantitative mutational analysis using MilliporeSigma internally developed software indicates the BT_0362 and BT_0364 base edited sample aTc100 agar plates harbor the expected C-to-T substitutions at the −17 position relative to the PAM for BT_0362 samples and the −18, −19 and −20 positions relative to the PAM in BT_0364 samples. Representative BT_0362 and BT_0364 samples are shown in (FIGS. 6A and B). These C-T substitutions result in an early stop codon introduction in both BT_0362 and BT_0364 base edited samples. The NT strain did not show any C-T substitutions in the targeted BT_0362 or BT_0364 regions after aTC induction.

This analysis software is called “SangerTrace”. It extracts each base signal peak value, based on Applied Biosystem's, Inc. format (ABI) file, and calculates mutation percentage by comparing “control” and “sample” of Sanger sequencing data.

### Example 3. CRISPR Base Editing in Other Bacteroides Strains

The NBU2 integrase recombination tRNA-ser sites (5′-CCTGTCTCTCCGC-3′ (SEQ ID NO: 2) are conserved and exist in many Bacteroides strains, including Bacteroides vulgatus, Bacteroides cellulosilyticus, Bacteroides fragilis, Bacteroides helcogenes, Bacteroides ovatus, Bacteroides salanitronis, Bacteroides uniformis, and Bacteroides xylanisolvens, based on published genome sequences. The inducible CRISPR-CDA cassette expressing a targeting guide RNA can be integrated on the chromosome of these Bacteroides strains, and targeted CRISPR-CDA C-to-T base editing of a specific gene in a strain expressing a targeting guide RNA can be achieved by treatment with aTc inducer (as described in Example 1). In case there is no NBU2 integrase sites on the chromosome of a specific species, these 13 base-pair DNA sequences can be readily inserted on the chromosome via recombination (e.g., Cre//oxP) or allelic exchange as described in the art to enable chromosomal CRISPR-CDA integration and targeted gene base editing.

### Example 4. CRISPR Base Editing of Bacteroides in Mouse Gut

Targeted, inducible CRISPR-CDA C-to-T base editing of specific Bacteroides species mouse gut in situ can be carried out by integrating a CRISPR-CDA cassette expressing a guide RNA targeting a species specific protospacer sequence onto the chromosome of its genome mediated by NBU2 integrase via bacterial conjugation. In an exemplary case, the mouse is a gnotobiotic animal colonized with one or more Bacteroides derived from a mammalian gut microbiota, including human. The aTc inducer can be applied at a specific point of time to the mouse gut, resulting in targeted mutation or inactivation of a specific gene in a species of the gut microbiota.

What is claimed is:

1. A protein-nucleic acid complex comprising an engineered RNA-guided nucleobase modifying system in association with a chromosome of a bacterial cell, wherein the engineered RNA-guided nucleobase modifying system is targeted to a specific locus in the chromosome of the bacterial cell, and the chromosome of the bacterial cell encodes an HU family DNA-binding protein comprising an amino acid sequence with at least 50% sequence identity to SEQ ID NO: 1.

2. The protein-nucleic acid complex of claim 1, wherein the engineered RNA guided nucleobase modifying system comprises (i) a CRISPR system comprising a CRISPR protein and guide RNA (gRNA) and (ii) a nucleobase modifying enzyme or catalytic domain thereof, wherein the CRISPR protein is a nuclease deficient variant or a nickase.

3. The protein-nucleic acid complex of claim 2, wherein the CRISPR system is a Type I CRISPR system, a type II CRISPR system, a type III CRISPR system, a Type IV CRISPR system, a type V CRISPR system, or a type VI CRISPR system.

4. The protein-nucleic acid complex of claim 2, wherein the CRISPR protein is Cas9, Cas12, Cas13, Cas14, or CasX.

5. The protein-nucleic acid complex of claim 2, wherein the gRNA is a dual molecule gRNA comprising a CRISPR RNA (crRNA) and a transacting crRNA (tracrRNA).

6. The protein-nucleic acid complex of claim 2, wherein the gRNA is a single molecule gRNA comprising a fused hybrid of a CRISPR RNA (crRNA) and a transacting crRNA (tracrRNA).

7. The protein-nucleic acid complex of claim 2, wherein the nucleobase modifying enzyme or catalytic domain thereof is chosen from cytidine deaminase 1 (CDA1), cytidine deaminase 2 (CDA2), activation-induced cytidine deaminase (AICDA), apolipoprotein B mRNA-editing complex (APOBEC) family cytidine deaminase, APOBEC1 complementation factor/APOBEC1 stimulating factor (ACF1/ASF) cytidine deaminase, cytosine deaminase acting on RNA (CDAR), cytosine deaminase acting on tRNA (CDAT), tRNA adenine deaminase, adenosine deaminase, adenosine deaminase acting on RNA (ADAR), or adenosine deaminase acting on tRNA (ADAT).

8. The protein-nucleic acid complex of claim 2, wherein the nucleobase modifying enzyme or catalytic domain thereof is a cytidine deaminase or catalytic domain thereof, and the engineered RNA guided nucleobase modifying system further comprises at least one uracil glycosylase inhibitor domain.

9. The protein-nucleic acid complex of claim 2, wherein the CRISPR protein is linked directly or via a linker to the nucleobase modifying enzyme or the catalytic domain thereof.

10. The protein-nucleic acid complex of claim 2, wherein the nucleobase modifying enzyme or catalytic domain thereof is linked directly or via a linker to an adaptor protein, and the CRISPR protein or the gRNA comprises an aptamer sequence capable of binding to the adaptor protein.

11. The protein-nucleic acid complex of claim 10, wherein the aptamer sequence is chosen from MS2/MSP, PP7/PCP, Com, N22, AP205, BZ13, F1, F2, fd, fr, GA, ID2, JP34, JP500, JP501, KU1, M11, M12, MX1, NL95, PRR1, ϕCb5, ϕCb8r, ϕCb12r, ϕCb23r, Qβ, R17, SP, TW18, TW19, VK, or 7s.

12. The protein-nucleic acid complex of claim 2, wherein the engineered RNA guided nucleobase modifying system comprises a nuclease deficient Cas9 or Cas12a variant linked to a cytidine deaminase or catalytic domain thereof.

13. The protein-nucleic acid complex of claim 1, wherein the engineered RNA-guided nucleobase modifying system is expressed from a nucleic acid that encodes the engineered RNA-guided nucleobase modifying system and is integrated into the bacterial chromosome.

14. The protein-nucleic acid complex of claim 1, wherein the engineered RNA-guided nucleobase modifying system is expressed from a nucleic acid that encodes the engineered RNA-guided nucleobase modifying system and is carried on an extrachromosomal vector.

15. The protein-nucleic acid complex of claim 1, wherein the amino acid sequence of the HU family DNA-binding protein encoded on the chromosome of the bacterial cell has at least 55%, at least 60%, at least 65%, at least 70%, at least 75%, at least 80%, at least 85%, at least 90%, at least 95%, or at least 99% sequence identity to SEQ ID NO: 1.

16. The protein-nucleic acid complex of claim 1, wherein the bacteria is a Bacteroides species or a strain level variant thereof.

17. The protein-nucleic acid complex of claim 16, wherein the Bacteroides species or strain level variant thereof is chosen from B. thetaiotaomicron, B. vulgatus, B. cellulosilyticus, B. fragilis, B. helcogenes, B. ovatus, B. salanitronis, B. uniformis, or B. xylanisolvens.

18. A method for modifying at least one nucleobase in a chromosome of a target bacterial cell, the method comprising expressing an engineered RNA-guided nucleobase modifying system in the target bacterial cell, wherein the engineered RNA-guided nucleobase modifying system is targeted to a specific locus in the chromosome of the target bacterial cell and the engineered RNA-guided nucleobase modifying system modifies at least one nucleobase within the specific locus, such that expression of a gene comprising the specific locus is altered, modified, and/or inactivated, and wherein the chromosome of the target bacterial cell encodes an HU family DNA-binding protein comprising an amino acid sequence with at least 50% sequence identity to SEQ ID NO: 1.

19. The method of claim 18, wherein modification of the at least one nucleobase results in introduction of at least one single nucleotide polymorphism and/or at least one stop codon within the specific locus in the chromosome of the target bacterial cell.

20. The method of claim 18, wherein the engineered RNA guided nucleobase modifying system comprises (i) a CRISPR system comprising a CRISPR protein and guide RNA (gRNA) and (ii) a nucleobase modifying enzyme or catalytic domain thereof, wherein the CRISPR protein is a nuclease deficient CRISPR variant or a CRISPR nickase.

21. The method of claim 20, wherein the CRISPR system is a Type I CRISPR system, a type II CRISPR system, a type III CRISPR system, a Type IV CRISPR system, a type V CRISPR system, or a type VI CRISPR system.

22. The method of claim 20, wherein the CRISPR protein is Cas9, Cas12, Cas13, Cas14, or CasX.

23. The method of claim 20, wherein the gRNA is a dual molecule gRNA comprising a CRISPR RNA (crRNA) and a transacting crRNA (tracrRNA).

24. The method of claim 20, wherein the gRNA is a single molecule gRNA comprising a fused hybrid of a CRISPR RNA (crRNA) and a transacting crRNA (tracrRNA).

25. The method of claim 20, wherein the nucleobase modifying enzyme or catalytic domain thereof is chosen from cytidine deaminase 1 (CDA1), cytidine deaminase 2 (CDA2), activation-induced cytidine deaminase (AICDA), apolipoprotein B mRNA-editing complex (APOBEC) family cytidine deaminase, APOBEC1 complementation factor/APOBEC1 stimulating factor (ACF1/ASF) cytidine deaminase, cytosine deaminase acting on RNA (CDAR), cytosine deaminase acting on tRNA (CDAT), tRNA adenine deaminase, adenosine deaminase, adenosine deaminase acting on RNA (ADAR), or adenosine deaminase acting on tRNA (ADAT).

26. The method of claim 20, wherein the nucleobase modifying enzyme or catalytic domain thereof is a cytidine deaminase or catalytic domain thereof, and the engineered RNA guided nucleobase modifying system further comprises at least one uracil glycosylase inhibitor domain.

27. The method of claim 20, wherein the CRISPR protein is linked directly or via a linker to the nucleobase modifying enzyme or catalytic domain thereof.

28. The method of claim 20, wherein the nucleobase modifying enzyme or catalytic domain thereof is linked directly or via a linker to an adaptor protein, and the CRISPR protein or the gRNA comprises an aptamer sequence capable of binding to the adaptor protein.

29. The method of claim 28, wherein the aptamer sequence is chosen from MS2, PP7, Com, N22, AP205, BZ13, F1, F2, fd, fr, GA, ID2, JP34, JP500, JP501, KU1, M11, M12, MX1, NL95, PRR1, ϕCb5, ϕCb8r, ϕCb12r, ϕCb23r, Qβ, R17, SP, TW18, TW19, VK, or 7s.

30. The method of claim 20, wherein the engineered RNA guided nucleobase modifying system comprises a nuclease deficient Cas9 or Cas12a variant linked to a cytidine deaminase or catalytic domain thereof.

31. The method of claim 20, wherein the nucleobase modifying enzyme or catalytic domain thereof, the CRISPR protein, and the gRNA are expressed from at least one nucleic acid integrated into the chromosome of the target bacterial cell.

32. The method of claim 20, wherein the nucleobase modifying enzyme or catalytic domain thereof, the CRISPR protein, and the gRNA are expressed from at least one nucleic acid carried on an extrachromosomal vector

33. The method of claim 31, wherein the nucleic acid encoding the CRISPR protein is operably linked to an inducible promoter.

34. The method of claim 33, wherein the promoter inducing chemical is anhydrotetracycline.

35. The method of claim 18, wherein the amino acid sequence of the HU family DNA-binding protein encoded in the chromosome of the target bacterial cell has at least 55%, at least 60%, at least 65%, at least 70%, at least 75%, at least 80%, at least 85%, at least 90%, at least 95%, or at least 99% sequence identity to SEQ ID NO: 1.

36. The method of claim 18, wherein the target bacterial cell is a Bacteroides species or a strain level variant thereof.

37. The method of claim 36, wherein the Bacteroides species or strain level variant belongs to the phylogenetic group defined as B. thetaiotaomicron, B. vulgatus, B. cellulosilyticus, B. fragilis, B. helcogenes, B. ovatus, B. salanitronis, B. uniformis, or B. xylanisolvens.
